(19) United States
(12) Patent Application Publication
(10) Pub. No.: US 2011/0029087 A1
(43) Pub. Date: Feb. 3, 2011

(54) INTERVERTEBRAL PROSTHESES WITH COMPLIANT FILLER MATERIAL FOR SUPPORTING ADJACENT VERTEBRAL BODIES AND METHOD

(76) Inventors: Thomas T. Haider, Riverside, CA (US); Nicholas M. Cordaro, Vista, CA (US)

Correspondence Address: SNELL & WILMER LLP (OC), 600 ANTON BOULEVARD, SUITE 1400, COSTA MESA, CA, 92626, US

(21) Appl. No.: 12/936,062

(22) PCT Filed: Apr. 3, 2009
(86) PCT No.: PCT/US09/39550

§ 371 Date: Oct. 1, 2010

Related U.S. Application Data
(60) Provisional application No. 61/072,987, filed on Apr. 4, 2008.

(51) Int. Cl. A61F 2/44

(52) U.S. Cl. 623/17.16

(57) ABSTRACT

An intervertebral prosthesis and method includes an artificial interbody disc for engaging faces of adjacent natural vertebral bodies when a natural disc is replaced. Channels in the interbody disc can provide flexible characteristics to accommodate loads on the subject's spinal column. A bio-compatible filler material having a flexural stiffness of less than 12 GPa can encourage bone growth in the channels. The filler material may be inserted during an operation or pre-inserted in the disc channels during manufacturing of the disc.

### CROSS-REFERENCE TO RELATED APPLICATION

This application is a continuation-in-part application from PCT/US06/47902 filed on Dec. 14, 2006 and also claims priority from U.S. Provisional Application Ser. No. 61/072,987 filed on Apr. 4, 2008.

### BACKGROUND OF THE INVENTION

1. Field of the Invention

This application relates to a method and an interbody disc with compliant natural and/or artificial filler material for restoring spinal motion between vertebral bodies between which a natural spinal disc has been removed in whole or in part.

2. Description of Related Art

In the field of spinal surgery, many treatment options exist to treat spinal pain, nerve impingement and spinal instability where a natural disc has failed in whole or in part. One such treatment is the removal of a damaged disc and its replacement with an intervertebral spacer which promotes fusion of bone between the separated vertebral bodies. This type of procedure when successfully completed, will result in a large bone mass between the vertebral bodies which will stabilize the column to a fixed position. See FIG. 1 where adjacent vertebral bodies 7 and 8 have been bridged with a solid mass of fused bone 9. This procedure is hereinafter referred to as rigid fusion. Also, see U.S. Pat. No. 6,447,547 to Michelson which discloses a spinal disc spacer intended to be infused with a solid, relatively motionless mass like FIG. 1. However, the success of a rigid fusion procedure appears to be one of many causes of adjacent segment disease. The lack of motion and the transfer of energy through the rigid fusion forces the adjacent intervertebral structures to adjust to the higher loads and motions or fail. Adjacent segment disease occurs as they fail.

Ball and socket type disc arthroplasty devices have been tried for over 30 years. See U.S. Pat. Nos. 5,676,701 and 6,113,637. Their design rational is to allow motion in the hopes of reducing higher loads to adjacent structures. These have shown some success but also failures. A ball and socket type device requires no energy to rotate. Thus, the work absorbed by the device during rotation is zero. The rotation centers may be favorable at one specific instantaneous center of rotation present in a natural healthy disc, but is never correct nor favorable for all movements. This forces abnormal loads on adjacent structures. Materials needed for a stable ball and socket device are often very stiff or incompressible, thus any axial loads and especially shock loads through the device are almost completely transferred to the adjacent structures. A patient expecting a favorable outcome with a ball and socket lumbar disc arthroplasty device may find unfavorable results if repeated axial loads I shocks (along the spine axis) are a common occurrence.

U.S. Pat. No. 4,309,777 to Patil, discloses an artificial disc with internal springs intended to flex. The device relies solely on the internal springs to provide the mechanical flexing motion. U.S. Pat. No. 5,320,644 to Baumgartner, discloses a different type of a mechanical flexing device. This device uses overlapping parallel slits forming leaf springs, which may contact in abrupt load paths, yielding impact stress. U.S. Pat. Nos. 6,296,664, 6,315,797 and 6,656,224 to Middleton, attempt to solve the disadvantage of abrupt load paths with a device containing a pattern of slits to allow for a more continuous load path. Middleton's device further includes a large internal cavity defined by the exterior wall. The internal cavity may be packed with bone to rigidly fuse adjacent vertebral bodies or capped with opposing plugs which limit the device's motion. Middleton's devices are intended to have a continuous load path with no abrupt load stops. These devices must be sufficiently stiff to support the anatomical average and extreme loads, thus too stiff to provide soft fusion as defined hereinafter.

U.S. Pat. No. 6,736,850, to Davis, discloses a pseudoarthrosis device containing small (0.25 to 2 mm inner diameter), flexible, permeable material tubes as to allow fibrous ingrowth. This device is very soft and may collapse under normal loads and may likely not form bone within the small inner diameters.

See published application nos. US20060217809A1; US20060200243A1; US20060200242A1; US20060200241A1; US20060200240A1; and US20060200239A1. It is the apparent attempt of the intervertebral prosthetic discs disclosed in these latter publications, to restore full intervertebral motion. However, these devices, as a result of their design, may be soft and very flexible resulting in artificial discs capable of absorbing little energy when subjected to shock loads. Computer simulations and mechanical validations of discs obviously patterned after some of these designs showed that it takes minimal loads (e.g., less than about 5 lbs for the cervical and less than 20 lbs for the lumbar) to compress the devices. While the weight required to be supported by an individual's spinal column will, to a great extent, depend on the individual's size, the weight to be supported in the cervical, thoracic and lumbar regions, will range from about 5 to 30 lbs, 30 to 60 lbs and 60 to 150 lbs or more in the cervical, thoracic and lumbar regions, respectively. Computer simulations also demonstrated that the use of a spiral slot or slit extending from the outer to the inner wall and encircling the disc two or more times as is illustrated in some of the publications is probably the reason for this lack of stiffness. A device which is too soft, will fully collapse when the patient is vertical, allowing for no additional movement to absorb impact energy. These types of soft spring devices, believed to have a stiffness of about 2.0 newtons(N)/mm, for use in the cervical region, and about 22.0N/mm in the lumbar region. Some of the patents/publications do show a vertical hole in the device, but apparently it came about for manufacturing purposes not for functionality. These patents do not describe or imply an intended fusion.

Several of the above references disclose the use of mechanical springs or bellows as the means to separate adjacent vertebrae while providing movement therebetween during flexure and extension. Such spring arrangements, beside their other problems, such as fracture at attachment points to end plates, provide little shock and energy absorption capability because they either fully compress at normal loads, or fracture at high loads.

There is a need for an intervertebral disc replacement or spacer for simulating the motion and energy shock absorption characteristics of a natural disc. To this end our novel intervertebral disc and method relies on a combination of mechanical flexure elements and bone and/or soft tissue infiltration within the disc to accommodate such motion and compliant filler materials such as a mixture of natural bone and/or artificial material for infiltration within the disc to accommodate such motion and energy absorption.

### SUMMARY OF THE INVENTION

Overview

A desirable condition, which we term soft fusion, can be created between a patient's adjacent vertebral bodies in which the natural disc has failed in whole or in part by a) removing the failed disc or failed portion thereof; b) installing an artificial intervertebral disc between the two vertebral bodies; c) the disc providing one or more selected continuous or discontinuous channels of limited size for bone to form and fuse into one or more continuous or discontinuous struts between the vertebral bodies; d) the device being stiff enough to support the bodies in their natural spaced relationship while allowing limited motion and flexible enough to transfer sufficient energy to the bone struts to create one or more conditions of nonunion joints or pseudoarthrosis resulting in living nonrigid bone growth; and e) the disc being further arranged to limit its movement to an amount which is sustainable by the disc without resulting in fatigue failure during an anticipated lifetime.

The cortical/cancellous bone of a vertebrae, particularly in the lumbar region, is very stiff. For example, a vertebral body 30 mm in diameter with cortical bone around the outer 5 mm and cancellous bone (softer bone) on the inner area, which is 25 mm in height, will have an axial stiffness of approximately 235,000 N/mm or 235 KN/mm. The stiffness (axial) of a disc enabling soft fusion in accordance with the present invention should be between about 50 to 4000 N/mm, preferably within the range of about 200 to 1500 N/mm and most preferably between about 400-800N/mm. The size of the bone accommodating channel(s) should occupy about 10-35% (or less) and preferably about 12% to 25% of the total area of the disc facing the vertebral body to be supported.

A condition of soft fusion is illustrated in FIG. 2 where a centralized bone strut 6 of limited dimensions is allowed to form within an open core or bone channel 17 of an interbody disc or spacer 10 to be described in more detail in connection with FIGS. 3-10.

The bone strut 6, extending between the vertebral bodies, has formed regions of pseudoarthosis or nonunion locations 6a. The nonrigid bone struts along with the mechanical properties of the artificial disc accommodate additional energy absorption with increased movement per given load simulating, to a significant extent, the performance of a natural disc.

Preferably in addition to the inclusion of an open continuous or discontinuous core(s) 17 to accommodate the bone strut(s) the spacer will include generally horizontally oriented tissue accommodating channels (“tissue channels”) 22, 24 to promote vascularization and fibrous tissue ingrowth. FIG. 2a illustrates vascularization taking place within the tissue and bone channels. We refer to a disc which enables soft fusion as well as accommodates soft tissue infusion such as is demonstrated in FIG. 2 as a hybrid disc or device.

The added advantage of tissue channels in conjunction with the bone strut forming channel(s) is that upon each loading and unloading cycle of the spine, nutrients and cellular waste will be pumped through tissue channels forming fibrous tissue within the tissue channels (vascularization). The nutrients and cellular waste are also pumped in and out to the bone strut(s). The disc may be “tuned” to match the deflection per load ratios to that of a natural healthy disc. The additional benefit of the soft tissue vascular areas (or bone void areas) is that soft tissue provides little initial resistance to compression but provides increasing resistance to an increasing compressive load. The natural disc is also softer at lower compressions than at higher compressions (axial or bending). A soft fusion device, infiltrated with adequate soft tissue in the tissue channels or voids will produce a device which is nearly as soft as the implanted device or natural disc, when subjected to light loads, and then become stiffer with increased compression or bending, just as a natural disc will. This unique ability of a soft fusion device with applied vascular cellular inputs promotes a device which will closely mimic a natural healthy disc embracing the ability for the soft tissue to heal due to vasculization or to fuse upon a lack of device motion due to non use or device collapse or flexural element(s) failure.

A soft fusion device may take on many different forms and structures which will be as individualized as the anatomical location, desired outputs, and designer preferences but encompass the spirit of the invention. Obviously, the device must have a stiffness less than that of bone, but sufficient to maintain the supported vertebrae in a desired spaced relationship when the spine is subjected to light loads and flexible enough to transfer sufficient energy to bone strut(s) to create nonunion joints 6a when the spine is subjected to additional loading. Bone growth between the vertebral bodies outside of the selected bone accommodating channels is to be inhibited by limiting the available void volume, orientating the voids in a direction generally tangent to load paths, adding cellular inputs to specific void areas, filling the voids with a fluid or softer material and/or other means. The bone channels shown in this application are generally vertical and generally continuous. This is not a requirement for a soft fusion device. The device may have multiple channels in varying directions which do not need to be continuous. A discontinuous bone channel or an interrupted channel may extend ⅓ the total device height from one vertebral body and ⅓ the total distance from the opposing vertebral body and the device may be interrupted within the middle ⅓ of the device, for example. A channel extending at an angle from the endplate, at 60 degrees from vertical for example, may be useful to in allowing for more axial compression than a vertical channel. All these variations are allowable and in the spirit of a soft fusion device.

It may be possible for an artificial spinal prosthesis or disc to accomplish the same degree of limited motion, load dampening, and energy absorption of a soft fusion device but without the living bone struts (and preferably soft tissues layers) created by soft fusion, it will not have the unique ability to adapt to the patient's loading conditions, repair itself when broken, and have the unique ability to fully support the vertebral column in the unlikely event that the underlying interbody disc fails.

It is to be noted that the creation of a soft fusion state after the installation of a soft fusion hybrid device, in accordance with this invention, is dependent upon a patient's level of activity. For example, if a patient is sedimentary, i.e., moves very little, the bone formed with the channel(s) will become dense and rigid limiting the motion and energy absorption while protecting the spinal column stability. If the patient is more active, i.e., subjecting the struts and the device to additional loads, e.g., walking, lifting, etc., the bone core(s) will be less solid, i.e., fractured, not fully formed and/or infiltrated by soft tissue, allowing for more motion and energy absorption. This type of soft fusion/hybrid device will be able to change throughout the life of the patient, just as the body is able to remodel for given inputs. If the mechanical dampening and flexible members of a soft fusion device fatigue, crack and fail, the device will slightly collapse. The collapse will limit the motion and eliminate the dampening action of the device thus transferring the energy to the supporting bone strut(s), promoting additional bone fusion and support.

Mathematical Rational

The theory behind soft fusion may be best understood by analyzing only the differences between a soft and rigid fusion rather than attempting to analyze actually true loads, deflections, and energy absorption capabilities. This is done by starting with basic equations.

Axial Deflection (δ) in the cephalic/caudal direction is equal to,

$$δ = \frac{PL}{AE} , \qquad ( Eqn . 1 )$$

where P is the applied force, L is the length of the strut (disc height), A is the cross-sectional area and E is the modulus of elasticity.

Bending curvature

$$( \frac{1}{ρ} )$$

either in flexion/extension or lateral bending is equal to:

$$\frac{1}{ρ} = \frac{M}{EI} \qquad ( Eqn . 2 )$$

where M is the applied bending moment and I is the moment of inertia.

Soft fusion works by displacing under applied forces more than possible with a ridged fusion. Strain energy (U) is defined as the energy uptake or energy absorbed by the deformation of the material by the applied load or:

U=∫₀^{x1}P*dx (Eqn. 3)

where P in an applied force and the integral of x from 0 to x1 is the deformation. Deformation noted in equations 1 and 2 may be inserted into equation 3 to determine the actually strain energy.

Many assumptions must be made to analyze the forces and deflections through the vertebral column and associated structures in order to accurately determine strain energy or energy absorption. However, the validity of soft fusion may be proven by simply comparing the variables unique between soft and rigid fusions. For the abovementioned device these are 1) the cross-sectional area of the bone strut verse the cross-sectional area of a rigid fusion 2) the presence or absence of the device in conjunction with the bone strut and for sake of comparison to arthroplasty ball and socket devices, 3) the modulus of elasticity.

To first look at the axial energy absorbed with the first set of variables we only need to define the cross-sectional area of a soft fusion as approximately 0.785 cm̂2 and the cross-sectional area of a rigid fusion as 15.4 cm̂2. These are typical cross-sectional areas seen within the lumbar region. By then setting the strain energy of a soft fusion to U_{S} and that of a rigid fusion to U_{R} the relation between the two becomes:

$$\frac{{U}_{s}}{{U}_{r}} = \frac{{\int }_{0}^{δ n} P * δ r}{{\int }_{0}^{δ r 1} P * d δ r} \qquad ( Eqn . 4 )$$

With equal assumptions to both soft and rigid fusions and with all variables except the cross-sectional areas equal, equation 4 becomes:

$$\frac{{U}_{s}}{{U}_{r}} = \frac{\frac{1}{As}}{\frac{1}{Ar}} = \frac{Ar}{As} = \frac{15.4 {cm}^{2}}{.785 {cm}^{2}} - 19.6 \qquad ( Eqn . 5 )$$

In other words, a fully formed soft fusion bone channel will absorb 19.6 times more axial energy than a rigid fusion based solely on the area of available bone. The soft fusion device will reduce this number to some degree, depending on the stiffness of the actual device. Such constricted bone growth should not fully form in active patients or become fractured with high patient generated forces. When this occurs the presence of nonunions and fibrous tissue within the defined strut location(s) will only aid the soft fusion energy absorption capabilities by softening the hybrid bone, tissue, and implanted device creating a condition of a controlled pseudoarthrosis.

By neglecting the minimal effects of the Soft Fusion device and only comparing the bone strut to a cobalt chromium ball and socket device we see that the strain energy relationship in axial compression is approximately equal to:

$$\begin{matrix}\frac{{U}_{SoftFusion}}{{U}_{CoCr}} = \frac{\frac{1}{As * {E}_{bone}}}{\frac{1}{{A}_{disc} * {E}_{COCR}}} \\ = \frac{{A}_{disc} * {E}_{COCR}}{As * {E}_{bone}} \\ = \frac{15.4 {cm}^{2} * 220 GPa}{.785 {cm}^{2} * 17 Gpa} \\ = 253\end{matrix} \qquad ( Eqn . 6 )$$

As seen in equation 6, a cobalt chromium articulating device is extremely poor at absorbing axial impacts.

Similar bending calculations are currently omitted because of their redundancies to this application but would show similar results.

Suitable Intervertebral Disc Structure for Enabling Soft Fusion

A preferred intervertebral motion restoring disc for supporting adjacent vertebral bodies in their natural spaced relationship after a natural disc has been partially or wholly removed in accordance with the present invention has upper and lower surfaces for engaging the faces of the vertebral bodies to be supported and a support structure between the surfaces having a stiffness within the range previously discussed. The disc defines one or more generally vertically oriented continuous or discontinuous bone growth channels of limited cross-sectional area enabling bone struts to form therein extending at least partially and preferably completely between the bodies. The disc (with its stiffness characteristics) and the resulting bone strut or struts are arranged so that predetermined axial and/or bending loads thereon, e.g., normal loads, loads associated with standing or walking, will not fully compress the disc allowing a narrowing of the distance between the supported bodies during normal motion and create one or more pseudoarthrosis or fibrous nonunion locations along the length of the strut(s) to provide soft fusion thereby limiting a complete rigid strut formation. The disc further fully compresses at predetermined excessive forces in order to protect the flexural members of the device from overloading and failure. The unique combination of one or more pseudoarthrosis bone struts and the mechanical disc supporting structure results in the condition of soft fusion as previously discussed. Such controlled and limited fusion, i.e., soft fusion, provides limited motion, both translational and rotational and energy and shock absorption characteristics surpassing that of a rigid fusion while preserving vertical column stability.

First, vertebral column stability is particularly important in that it prevents disc induced or allowed kyphosis and scoliotic curvatures as seen with ball and socket type devices. Some prior art articulating devices will often settle into a fully rotated position when the soft tissue is unable to stabilize the spinal column. A soft fusion disc provides a force towards the central position assisting to stabilize the spinal column. Second, disc stability is important in that the continuous or discontinuous bone channels will likely form some degree of bone with soft tissue infiltration. This will greatly aid in preventing device expulsion, a failure mode seem with other non-fusion devices.

One such intervertebral disc acceptable for providing soft fusion and particularly designed for anterior insertion in the lumbar/thoracic region includes a pair of end plates (or layers) with each end plate having an outer intervertebral engaging surface for buttressing against a respective vertebral body and an inner surface. A plurality of interleaved first and second axial dampening plates (or layers) are sandwiched and secured between the inner surfaces of the end plates.

Each of the individual dampening plates define a peripheral outer wall and an inner generally cylindrical open bone accommodating core aligned along a longitudinal axis which will be generally aligned with the patient's spinal column when installed. Every other pair of axial dampening plates may be bonded, e.g., welded, together adjacent the inner core (or machined) leaving a generally planar space therebetween extending outwardly from the bonded area beyond the outer walls. The remaining pairs of axial dampening plates may be bonded, e.g., by welding, together along their peripheral walls (or machined) leaving a generally planar space therebetween extending from the bonded area to the open core. This arrangement provides alternating spaces extending from the core outwardly and from the peripheral walls inwardly which allows the end plates and the vertebral bodies to which they are secured to have limited translational motion parallel to the longitudinal or spinal axis and limited pivotal motion about the axis while dampening both motions. The channels formed between the plates and particularly the channels extending inwardly from the peripheral wall will accommodate tissue infusion and function as tissue channels.

Preferably the dampening plates are provided with one or more flexion slots between the outer peripheral walls and the inner cores to provide increased flexing action. The periphery of plates preferably follow the contour of the disc which they are to replace, e.g., an outer, generally convex, peripheral wall merging with a generally concave inner wall. As an option, a rotational dampening subassembly, to provide limited rotational motion between the end plates, can be inserted into the sandwiched axial dampening plates. Such assembly comprises an inner generally circular planar torsional dampening spring member with a helical slot, mounted between upper and lower torsional plates so that one of the torsional plates can rotate through a limited angle relative to the other. Alternatively, the spacer may be formed with about a 1½ turn or helical slot extending from the exterior wall to the central core(s) eliminating the interleaved plate construction as will become apparent in reference to the appended drawings.

The plates may be made of a suitable biocompatible material such as a titanium, cobalt or stainless steel alloy and or super elastic metals, e.g., nitinol, which in the sandwiched assembly, has sufficient strength and flexibility (stiffness) to withstand the anticipated stresses while providing the desired motion requirements to allow nonrigid bone struts to form within the open core.

In one method of construction the assembly is built plate by plate (or layer by layer) with the individual plates joined by diffusion, laser or electron beam welding or perhaps with a mechanical interference fit only.

The assembly may be constructed in various configurations adapted to site specific in vivo locations such as anterior, anterior lateral, lateral, lateral posterior or posterior spinal interbodies, interspinous dampening spacers, interconnecting pedicle screw dampening members or other posterior element stabilization devices.

An intervertebral disc particularly designed for the cervical region of the spine is formed with upper and lower surfaces for engaging the respective vertebrae faces to be supported and a generally elliptical partially obstructed open core for accommodating the formation of one or more bone struts. The spacer includes generally planar semicircular soft tissue integration channels extending inwardly from a peripheral wall to a location short of the open core. The tissue channels are interleaved with planar channels extending outwardly from the core to a location short of the peripheral wall.

While providing various examples of intervertebral prosthetic discs and a method for accommodating the creation of soft fusion within the discs advances the state of this art, we now propose improvements to provide a superior prosthesis and method by filling the channels defined in the intervertebral prosthetic discs with a material which is less stiff than typical cortical bone including some cancellous bone used in the prior art devices. For example, a filler material having a flexural stiffness less than 10-12 GPa will improve the load compliance and flexibility of the intervertebral prosthesis.

Where a cortical/cancellous bone blend is to be used as the filler material, cancellous bone, which has a GPa of the order of 4 GPa, should comprise at least the predominate, if not, the sole constituent of the blend. This flexural stiffness is reported to be the average for cancellous bone. The use of such softer filler materials in the discs will allow for a more compliant and energy absorbing device even in the absence of a nonunion joint or pseudoarthosis. A softer filler material will in effect alleviate the need for a nonunion or pseudoarthosis by the formation of a more compliant yet stable fusion.

An interbody disc, in accordance with the present invention, has (a) upper and lower surfaces for engaging the faces of the adjacent vertebral bodies between which a failed natural disc has been partially or wholly removed, (b) an exterior wall and one or more generally vertically oriented continuous or discontinuous channels (c) a sufficient stiffness to support the separated vertebrae in substantially their naturally spaced relationship while allowing limited motion and flexibility when subjected to a predetermined load to alter the distance between the vertebrae and thereby transfer load and energy to the any material filling the channels or voids and (d) a bio-compatible filler material disposed within the channels, the filler material being compliant and softer than cortical bone, e.g., having a flexural stiffness of less than about 10-12 GPa. The filler material combined with the device characteristics—will dampen the loads and energy transfer prior to the device contacting on the internal stops which will then in turn prevent fatigue failure.

Where human bone is selected as the filler material, cancellous bone is the first choice. As a second choice cancellous and cortical bone can be blended with cancellous bone being the predominate portion of the blend such as a ratio of cancellous to cortical bone within the range of about 80% to 20% and preferably about 60%+ to 40%.

Other naturally harvested materials (either from the patient, a donor or an animal) suitable for use in the blend can include any substances softer than bone, such as portions of the removed disc. Morselized bone or bone weakened by gamma sterilization is more compliant then cortical bone and may also be useful as a filler material.

Bone graft substitutes, such as demineralized bone matrix (DBM), calcium sulfate dehydrate (CSD) ceramic-based bone graft extenders, are believed to be satisfactory filler materials. These will have a very low flexural modulus to allow device bending characteristics but will resist compressive forces when contained in a generally vertical channel. Recombinant Human Bone Morphogenetic Protein (rhBMP-2) liquid, Epidermal Growth Factor (EGF) liquid, Platelet Derived Growth Factor (PDGF), Fibroblast Growth Factors (FGFs), Parathyroid Hormone Related Peptide (PTHrp), Insulin-like Growth Factors (IGFs), and Transforming Growth Factor-Betas (i.e., TGF-B1), may also accompany suitable filler materials in order to induce a specific biological response such as bone or soft tissue activity. Another filler material candidate is polyetheretherketone (PEEK) with or without porosity. This synthetic material has mechanical properties very similar to those in cortical bone without porosity and very similar to cancellous bone when used with porosity. In addition, it is highly controllable and stable allowing the disc to be preassembled with the filler material at a factory site.

The method of the present invention entails a) providing a prosthetic disc as discussed above, b) filling the channels with the appropriate filler material, either at the surgical site or at a manufacturing site, c) removing the damaged or failed disc in whole or in part, and d) inserting the filled disc between the separated vertebral bodies.

The structure and function of an intervertebral disc for creating soft fusion and method for accomplishing this desired condition are explained in more detail in the accompanying description of the preferred embodiments taken in conjunction with the appended drawings wherein like components (or locations) are given the same reference numerals.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings.

FIG. 1 is a side elevational view of adjacent vertebral bodies separated by a conventional rigid fused bone mass;

FIG. 2 is a perspective view, partially in cross-section, of adjacent vertebral bodies separated by an interbody disc and a central bone strut containing fibrous nonunion locations forming a soft fusion disc;

FIG. 2a is an enlarged partial view of the disc of FIG. 2 showing vascularization taking place within the tissue/bone channels;

FIG. 3 is a perspective view of an anterior interbody disc in accordance with the present invention;

FIGS. 4 and 5 are plan and front views, respectively, of one of the outer or first dampening plates of the disc of FIG. 3;

FIGS. 6 and 7 are plan and front views, respectively, of one of the inner or second dampening plates of the disc;

FIG. 8 is a front elevational view of the assembled plates of FIGS. 4-7;

FIGS. 9 and 10 are top plan and front views, respectively, of the disc;

FIG. 11 is a cross-sectional view of the disc of FIG. 1 taken along lines 11-11;

FIG. 12 is a front elevational view of the disc showing articulated/pivotal motion between the end plates about the longitudinal axis;

FIGS. 13 and 14 are top plan views, respectively, of the upper and lower torsional plates of a torsional dampening subassembly;

FIG. 15 is a plan view of an inner torsional dampening spring member of the subassembly;

FIG. 16 is a front view of the assembled torsional dampening subassembly;

FIG. 17 is a cross-sectional view of the device of FIG. 3 incorporating the rotational dampening subassembly, taken along lines 11-11 of FIG. 3;

FIG. 18 is a cross-sectional view of the device of FIG. 3 incorporating the rotational dampening subassembly taken along lines 18-18;

FIG. 19 is the same cross-sectional view as FIG. 17 showing articulation of the device;

FIG. 20 is a perspective view, partially broken away, of a modified disc similar to the disc of FIG. 3 showing the migration of bone within the central core and soft tissue within the soft tissue channels or voids;

FIG. 21 is a cross-sectional view showing the disc in a vertically compressed mode;

FIG. 22 is a graph showing a typical moment versus rotation plot of a natural disc versus that of a computer model of the artificial disc of FIG. 20 when bone and tissue have penetrated the voids as illustrated in FIG. 20;

FIGS. 23 and 24 are perspective and side elevational views, respectively, of an alternative embodiment of a disc primarily designed for the cervical region;

FIGS. 25 and 26 are cross-sectional views taken along lines 25-25 and 26-26, respectively in FIG. 23;

FIGS. 27-29 are top plan, side elevational and cross-sectional views of other interbody discs for providing increased rotational mobility;

FIG. 29a is a cross-sectional view of the disc of FIGS. 27 and 28 as modified to eliminate the threaded connection and provide a gap between the exterior walls of the upper and lower sections;

FIGS. 30 and 31 are side elevational and cross-sectional views of another interbody disc;

FIGS. 32-34 are top plan, side elevational and cross-sectional views of another disc embodiment;

FIGS. 35-37 are top plan, side elevational and cross-sectional views of another disc embodiment;

FIGS. 38-42 are top plan, side elevational, bottom, end and cross-sectional views, respectively, of a base component of an alternative two-piece disc;

FIGS. 43-47 are top plan, side elevational, bottom, end and cross-sectional views of an upper component of the two piece disc;

FIGS. 48-51 are top plan, side elevational, bottom and cross-sectional views of the assembled two piece disc;

FIGS. 52-56 are top plan, side elevational, bottom end and cross-sectional views of an upper component of an alternative two-piece disc;

FIGS. 57-61 are bottom plan, side elevational, top, end, and cross-sectional views of a base or bottom component of the two-piece disc;

FIGS. 62-64 are side elevational, end and cross-sectional views of the assembled two piece disc;

FIGS. 65-68 are top plan, side elevational, end and cross-sectional views of another embodiment of an anterior disc;

FIGS. 69-72 are top plan, side elevational, end and cross-sectional views of a posterior disc in accordance with the invention;

FIG. 73 is a perspective view of two of the discs of FIGS. 69-72 placed on the exposed face of a lower vertebral body;

FIGS. 74-78 are top plan, side elevational, front end, rear end and cross-sectional views of an alternative posterior disc;

FIGS. 79 and 80 are a perspective view and a cross-sectional view respectively, taken along the length of the disc, insitu between vertebral bodies, with bone growth shown;

FIG. 81 is a cross-sectional view, insitu between vertebral bodies of a sheep with bone growth shown;

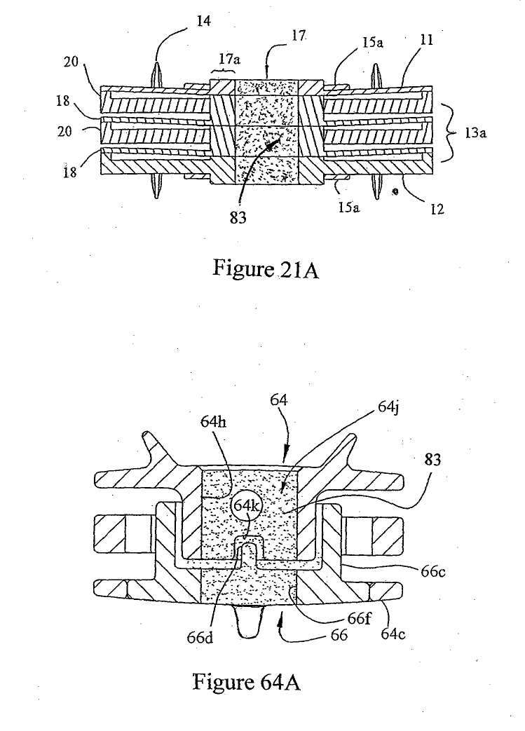

FIG. 21A is a cross-sectional view showing the disc of FIG. 21 with filler material;

FIG. 64A is a cross-sectional view showing the disc of FIG. 64 with filler material; and

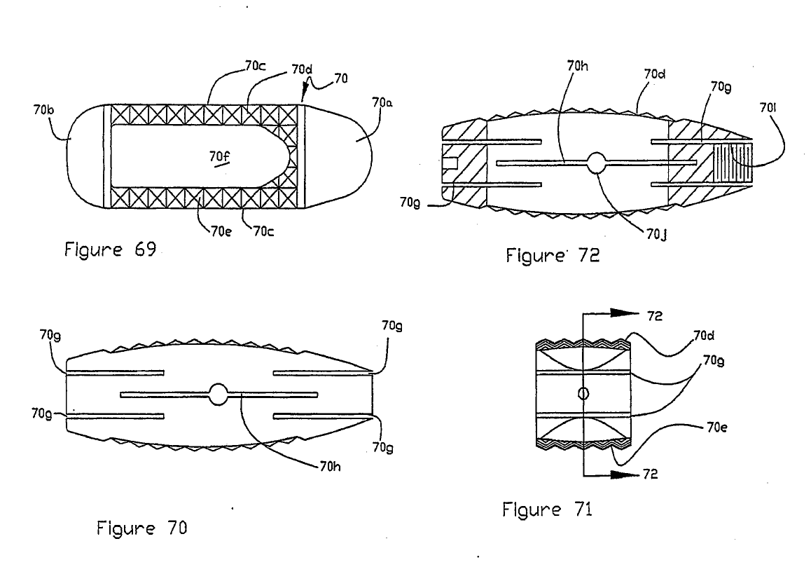

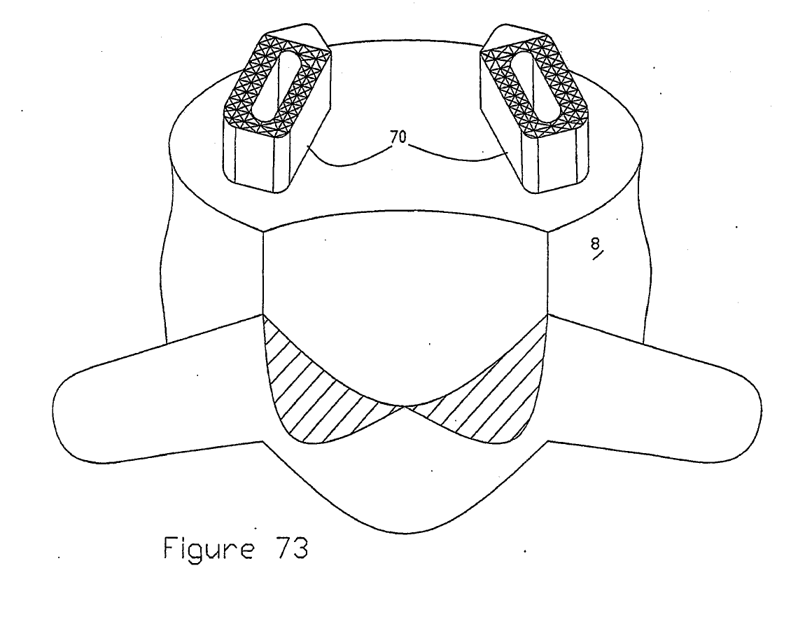

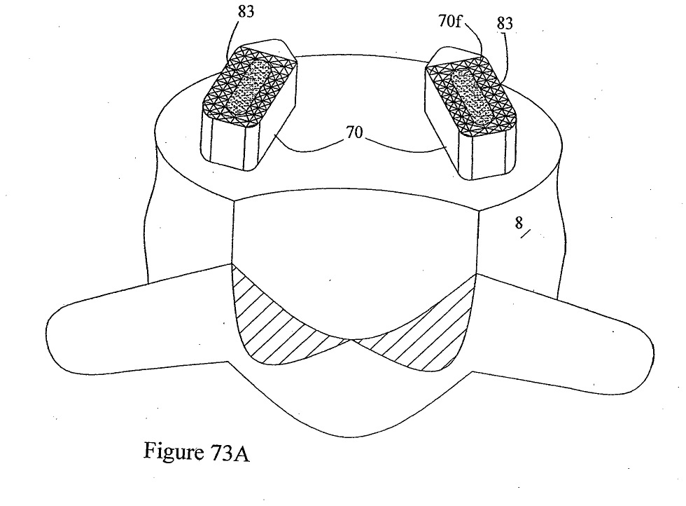

FIG. 73A is a perspective view of the discs of FIGS. 69-72 with filler material.

### DETAILED DESCRIPTION OF THE PREFERRED EMBODIMENTS

Reference will now be made in detail to the preferred embodiments of the invention which set forth the best modes contemplated to carry out the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and circuits have not been described in detail as not to unnecessarily obscure aspects of the present invention.

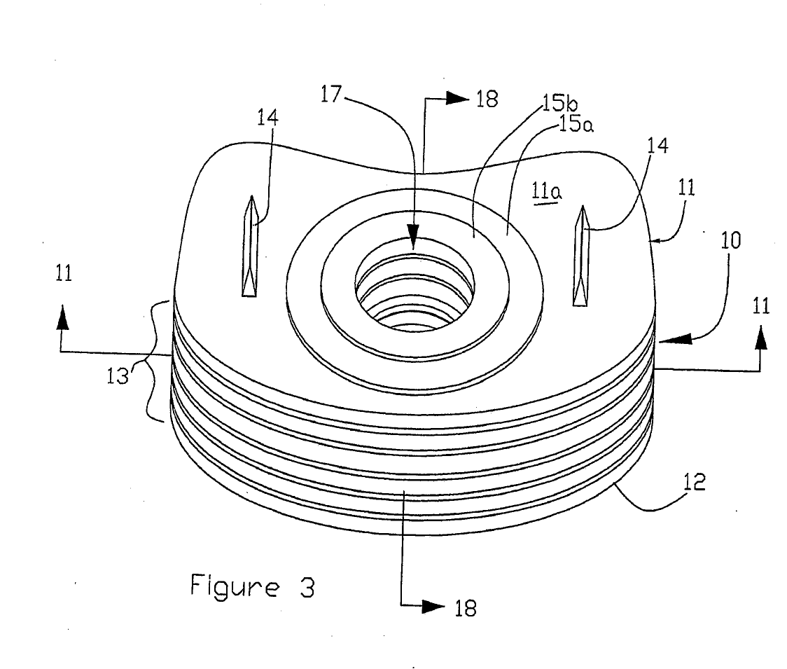

Referring now to FIG. 3 an upper end plate 11, providing an outer or cephalad surface 11a for buttressing against an upper vertebral body, is joined to a lower end plate 12, providing an outer or caudal surface 12a (not shown in FIG. 3). A group of interlocking interior plates or layers, collectively referred to as 13, are disposed between and joined to the inner surfaces 11b and 12b to form an anterior interbody device or artificial disc. See FIG. 11. The outer surfaces 11a and 12a of the end plates may be provided with an array of mechanical locking features such as the keels 14 or alternative geometric features and fixation rings 15a and 15b. The fixation rings may be constructed of an osteointegrative porous material which abut the edge of a hollow core or bone channel 17. As discussed previously, the core 17 accommodates bone growth to form a continuous or discontinuous strut (with nonunion locations) adjoining the separated vertebral bodies.

The fixation rings are stepped to provide additional purchase against the vertebral end plates and to fill the convex surface of the adjacent vertebral end plate. The core may be packed with bone to accelerate the formation of the strut or other material. The core 17 and the interior plates layers 14 may be, but preferably are not, shielded from surrounding tissues to prevent tissue integration or device particulate wear explosion. Dacron or polytetrafluroethylene are preferred material to provide device shielding if desired.

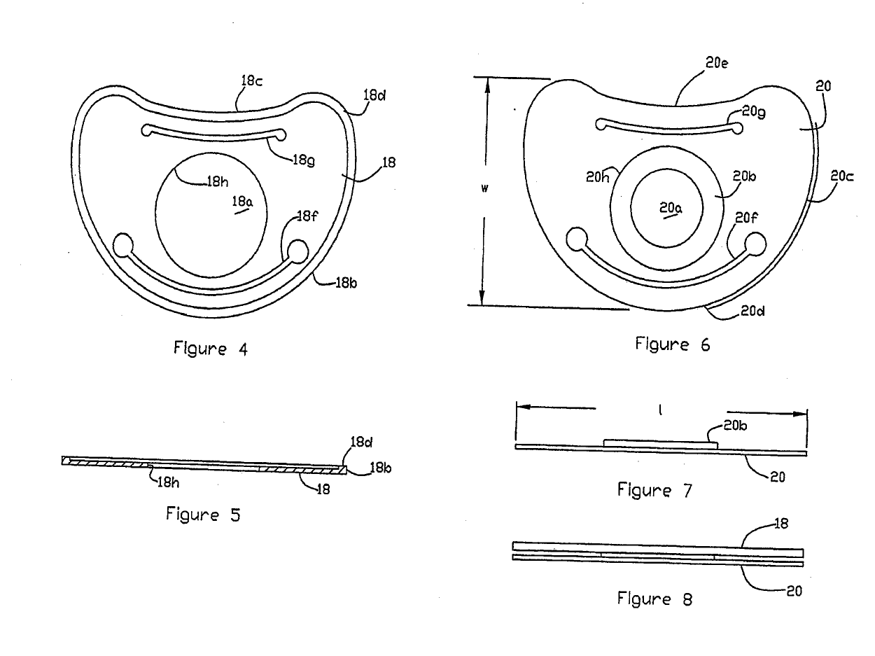

FIGS. 4 and 5 illustrate a first axial dampening plate 18 which forms one of the interior interleaved plates of the assembly or group 13. The plate 18 includes a central cylindrical opening 18a, a generally convex peripheral front wall 18b, merging with a generally concave back wall 18c, a protruding lip 18d, extending along the periphery, and optional front and back flexion slots 18f and 18g. The opening 18a is framed by a cylindrical wall 18h which is bonded to an inner shoulder of the second plate as will be explained.

FIGS. 6 and 7 illustrate a second axial dampening plate 20. The second plates are interleaved with the first plates and disposed between and joined to the inner surfaces of the end plates 11 and 12 to form the motion restoring intervertebral device of the invention. The second plate 20 includes a central opening 20a, an upwardly protruding inner shoulder 20b surrounding the opening, a peripheral wall 20c in the form of front and back walls 20d and 20e, respectively. The second plates include flexion slots 20f and 20g which align with slots 18f and 18g in the assembled device.

FIG. 8 illustrates the first and second plates in an assembled condition with the first plate's inner surface 18h of the opening 18a being bonded to an outer surface 20h of the second plate's protruding lip 20b.

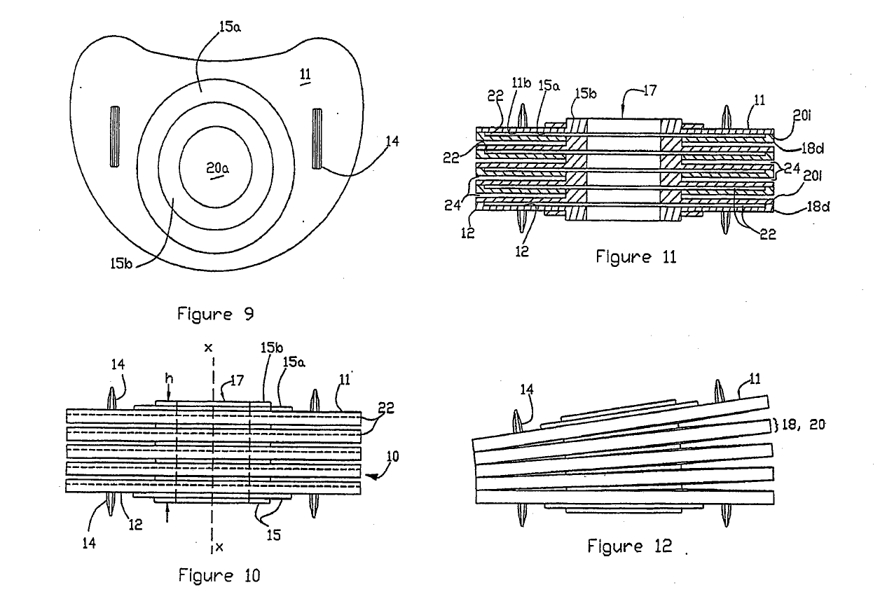

FIGS. 9 and 10 represent a top plan view and a front side view, respectively, of the assembled intervertebral device. It is to be noted that the second plates, when provided with an outer ring 15a, can be used as the end plates, as is shown in the cross sectional view of FIG. 10. The horizontal dashed lines represent the bond or weld between the peripheral walls of the first and second plates although the welds would generally not be visible in the finished device. X-X represents the longitudinal axis of the device. The term axial loads, as used herein, refers to loads or forces directed along the X-X axis. The aligned openings 20a of the second plates represent the open core 17.

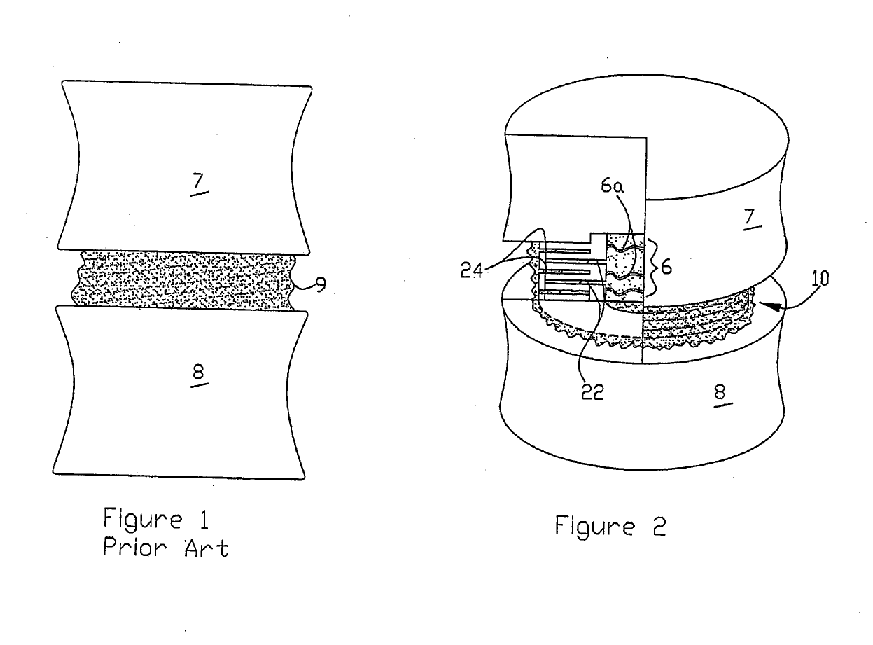

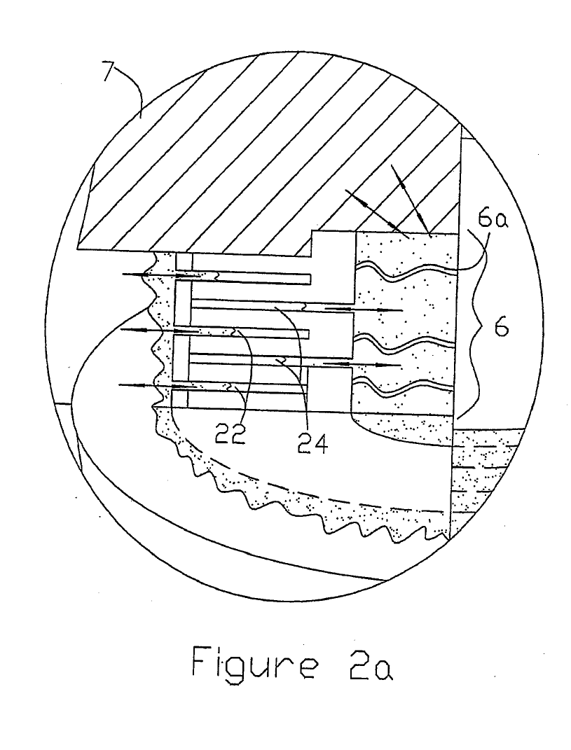

Referring now to FIG. 11, a cross sectional view taken along line 11-11 of FIG. 1, the peripheral lip 18d of the first plates are bonded to a peripheral annular surface 20i on one side of the second plates leaving a generally planar space 22 extending inwardly from the bond or weld between 18d and 20i to the open core 17 as is shown in FIG. 9. The annular surface 18h, surrounding the opening 18a of the first plate, is bonded to the outer surface 20h of the next second plate to provide a generally planar space 24 extending outwardly from the bond (i.e., surface 20h) to the outside of the device as illustrated. This pattern is repeated with the plates being assembled one plate at a time until all plates are stacked and welded. If the device is diffusion welded, all plates may be stacked and welded at one time. The spaces 22 and 24 are preferably left open as shown and serve to allow the infusion of soft tissue as previously discussed.

The intervertebral disc, with its interleaved plates, has motion yet sufficient stiffness or strength to support the vertebral bodies (7, 8) in their natural spaced relationship while allowing limited motion and dampening the load applied to the bodies. When the separated vertebrae are subjected to normal loads, such as would be experienced by a person standing or walking, the plate will not be fully compressed allowing a narrowing of the distance between the vertebral bodies causing the bone strut formed in the core 17 to fracture or form fibrous nonunion joints at one or more locations along its length. Greater loads, such as jogging or lifting heavy objects, will further aid this process of promoting nonunions.

The fatigue life of the device is preserved by the internal spaces 22 and 24 as shown in FIG. 11 which spaces are preferably left open to accommodate soft tissue ingrowth therein forming a hybrid device. The individual plates or layers may flex, bend (and/or rotate with an optional torsional dampening subassembly to be described) as designed until they deflect to a point collapsing the internal spaces or preferably compressing tissue infused therein. Once the internal spaces are collapsed with or without tissue therein, the individual plate's movement will be stopped by an adjacent plate. All plates or layers are designed so that movement within these internal spaces will not fatigue the material, thus preserving the fatigue life of the device.

FIG. 12 is a front view illustrating an assembled intervertebral device under going articulation.

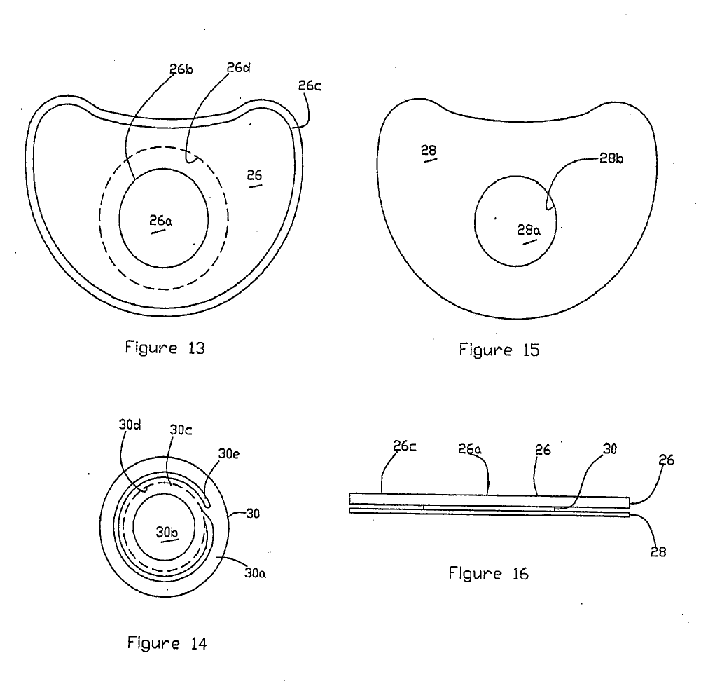

FIGS. 13 and 15 are top plan views of the outer plates 26 and 28 which together with a spring 30 (FIG. 14) form a torsional dampening subassembly shown in FIG. 16. Both plates follow the outside contour of the first and second plates and end plates. The upper plate 26 defines a central opening 26a (aligned along the longitudinal axis), a downwardly extending annular undercut 26b (shown in dashed lines), and an upwardly extending peripheral lip 26c (like the peripheral lip 18d). The lower plate 28 defines a central opening 28a surrounded by a surface 28b. A torsional spring member 30 includes peripheral area 30a, a central opening 30b, surrounded by an annular undercut 30c (shown in dashed lines) in FIG. 13 and with an undercut edge 30d, and a spiral slot 30e which allows limited rotation between the depending shoulder 30c and the peripheral area 30a.

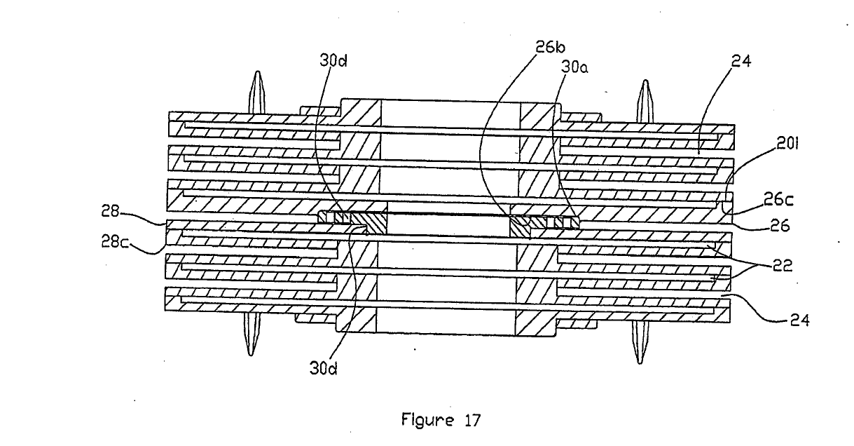

FIG. 16 shows the torsional dampening subassembly in its assembled form while FIG. 17 illustrates a front cross-sectional view of the device of FIG. 1 including the addition of the torsional dampening subassembly. The lip 26c of the upper plate is bonded to the second plate's peripheral lower surface 20i. The edge 26d of the undercut 26b is bonded to the peripheral area 30a of the spring member 30 with the surface 30d of the spring member 30 being bonded to the surface 28b of the lower plate 28. The lower peripheral surface 28c of the lower plate 28 is bonded to the peripheral lip of the first plate as shown.

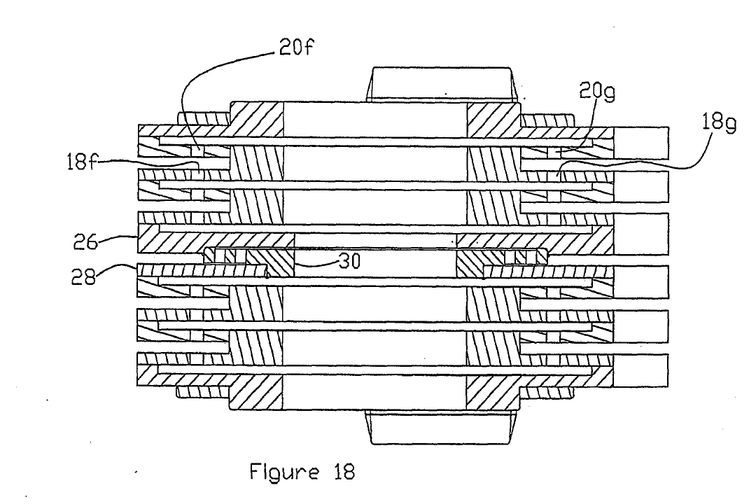

FIG. 18 is a side cross-sectional view of the device of FIG. 17 showing the flexion slots 18f, 18g, and 20f, 20g.

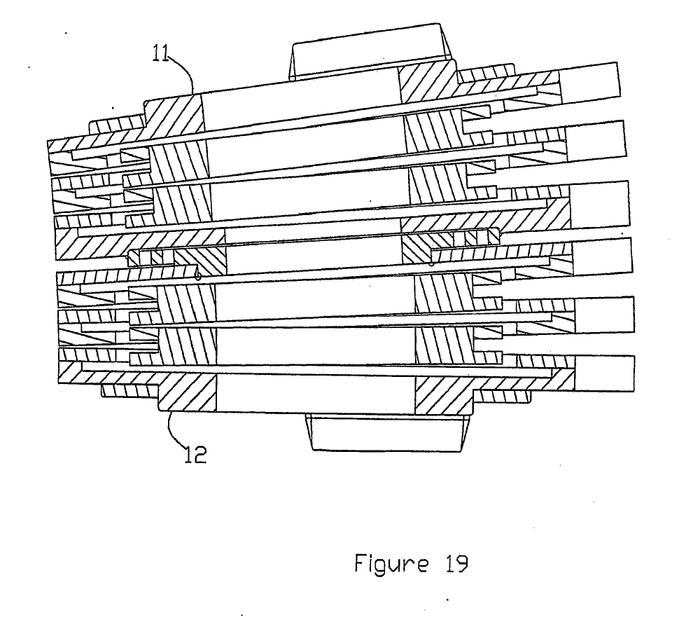

FIG. 19 is a side cross-sectional view of FIG. 17 illustrating articulated/pivoted motion between the end plates 11 and 12.

The overall height h (FIG. 10) of the intervertebral motion restoring device will depend upon its selected location and the patient's anatomy. As an example, h should be within the range of about 0.19 to 0.315 inches and 0.315 to 0.8 inches for use in the cervical or thoracic and lumbar regions, respectively.

As a further example for a height h of 0.565″ the spaces 22 and 24 (FIG. 5) are preferably 0.015 and 0.012 inches, respectively. Such a device may have a width w and a length t (FIGS. 4 and 5) of about 1.0 and 1.4 inches, respectively.

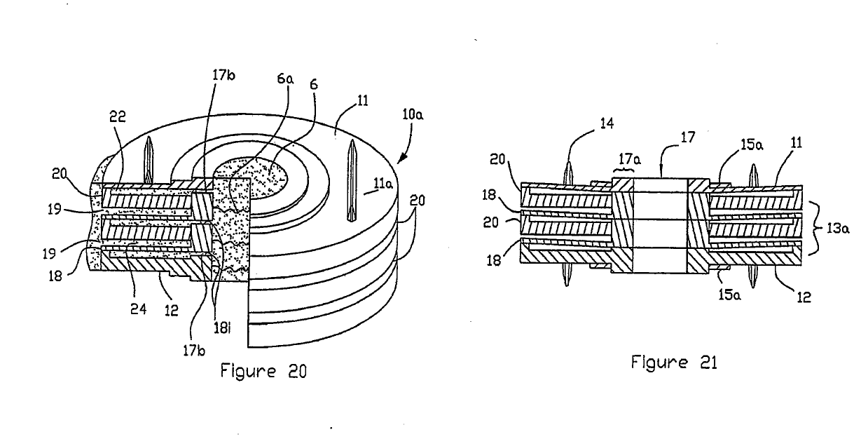

A slightly modified disc is shown in FIGS. 20 and 21 in which corresponding components are identified with the same numerals.

The difference between the intervertebral disc of FIGS. 20-21 and the disc described in FIGS. 3-12 (except for the number of intermediate plates 18, 20) is the addition of the extensions 18i of central portions of plates 12 and 18 which, when the spacer is in the unstressed condition, form gaps 17b in the central section. These extensions serve as a stop means to limit the compression and axial articulation of the support structure without completely closing the channels 22 and 24, preserving the fatigue life of the disc.

As an example, for a support structure 13′ having a height of about 5 mm, the gaps 17′b between the extensions 18i and the adjacent plates may be about 0.015″ while the channels 22 and 24 may have a height of about 0.020″. This difference in the dimensions of the gaps versus the height of the channels allows the spacer to be completely compressed (i.e., along the longitudinal axis) without completely closing the channels 22 and 24 by providing stop means, i.e., contact locations along the central section 17a, to accommodate abrupt loads and to alleviate fatigue failure which may otherwise occur as a result of repetitive loads. This also prevents complete soft tissue compression within the voids 22 and 24 and allows for additional disc bending when fully compressed.

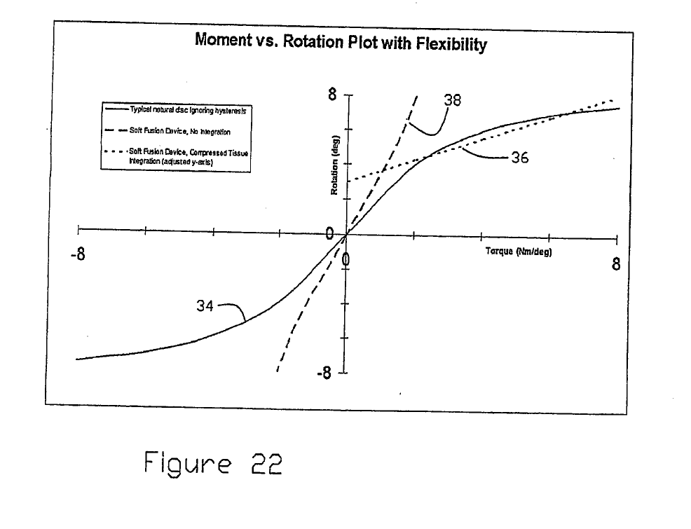

FIG. 20 illustrates the infiltration of soft tissue 19 within the channels between the plates and some migration of a bone strut 6 within the core 17 forming nonunions at locations 6a. The soft tissue infiltration in the large areas within the channels results in a nonlinear increase in stiffness of the spacer as the load is increased thereby simulating the response of a natural disc. The design of FIGS. 20 and 21, as well as the designs shown in subsequent figures have the ability to openly integrate with varying combinations and densities of bone and soft tissue, thus producing a hybrid device made of both inorganic (metal or polymer) and organic (cellular tissue and/or bone) materials. The advent of continuous or discontinuous bone struts through the device as discussed previously will yield a device which is stable, yet more flexible, than a device relying on rigid fusion, thus providing the capability of energy absorption. It is to be noted, however, that bone struts are not required to produce a positive result. Soft tissue, will in time, infiltrate the voids producing a device which will more closely mimic a natural disc. This will be explained in more detail in conjunction with FIG. 22.

FIG. 21 illustrates the disc completely compressed by a vertically oriented or axial load with the gaps 17′b closed. The spacer in such a collapsed mode will still accommodate a lateral bending action, i.e., about the longitudinal axis.

FIG. 22 is a graph showing lumbar-disc moment in Newton meters/degree verse rotation plot in degrees (around a horizontal axis) of a typical natural disc (curve 34), hysteresis ignored. This response of a natural disc is illustrated by Dr. Spenciner D. et al., in The multidirectional bending properties of the human lumbar intervertebral disc. Spine J. 2006 May-June 6(3):348-57. The slope of the curve at any point represents the disc flexibility. Curves 38 and 36 represent the theoretical response of a computer model of the intervertebral spacer of FIGS. 20 and 21. It is to be noted that the response of an actual mechanical disc made in accordance with this invention may vary from that shown by the curves. For small displacements and loads, soft tissue integration within channels 22, 24 will provide little resistance and thus the device will have a greater flexibility as shown by curve 38. As the motion and loads increase, the soft tissue will become increasingly compressed and strained. Due to soft tissues' nonlinear mechanical properties, the soft tissues will provide an increased degree of resistance with each increase in motion as is illustrated by curve 36. The actual hybrid Soft Fusion device flexibility curve will include a curve similar to curve 38 and transition to a curve similar to curve 36.

The area of the bone accommodating core 17 or cores should not exceed about 35% and preferably less than about 25%, (e.g., about 10-20%) of the total area of the disc facing the separated vertebral bodies, i.e., in a horizontal plane. The size of the disc and bone strut opening(s) therein will depend upon the size of the vertebral bodies to be supported. As an example, the total area of the openings should have a diameter, if circular, or equivalent dimensions if non-circular, within the ranges of 0.1 to 0.6, 0.1 to 0.7, and 0.2 to 0.7 inches in diameter for the cervical, thorax, and lumbar regions, respectively.

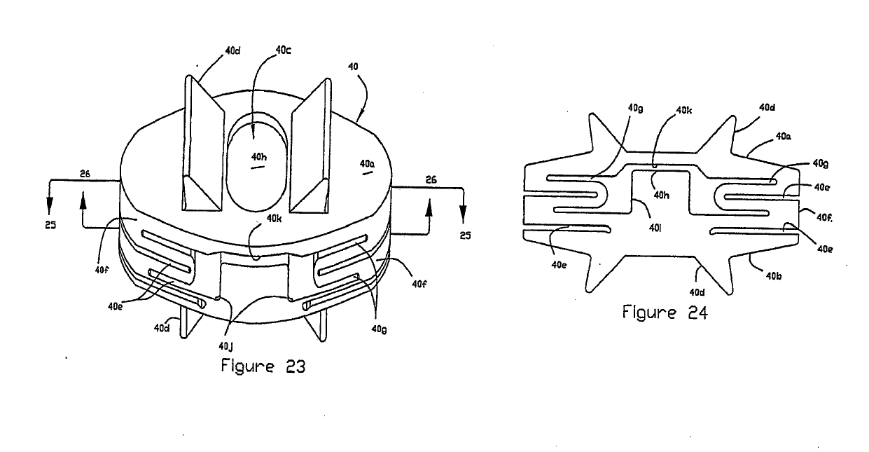

An alternative embodiment of an intervertebral or hybrid disc designed primarily for the cervical region, is illustrated in FIGS. 23-26 wherein the disc 40 is formed with upper and lower surfaces 40a and 40b, respectively and a central elongated generally elliptical open core 40c, partially obstructed by a bone integration diversion plate or beam 40h to be described. Keels 40d extend outwardly from the upper and lower surfaces to aid in securing the spacer between the supported vertebral bodies. The spacer includes generally planar semicircular soft tissue integration channels 40e extending inwardly from the exterior or peripheral wall 40f to a location short of the open core 40c. Generally semicircular tissue integration channels 40g are interleaved with the channels 40e and extend outwardly from the core 40c to a location short of the exterior wall. The centrally located bone diversion beam 40h extends laterally across the core 40c below the upper surface 40a as shown more particularly in FIGS. 9 and 10.

The beam is held in place by downwardly extending legs 40i which are formed with or otherwise secured to the lower peripheral wall at 40j (FIG. 23). The ends of the beam 40h are arranged to abut the opposed ends 40k of the top of the spacer at the ends of the open core to limit the compression (and vertical articulation) of the spacer when subjected to excessive loads. It is to be noted that the number and configuration of the tissue integration channels may vary.

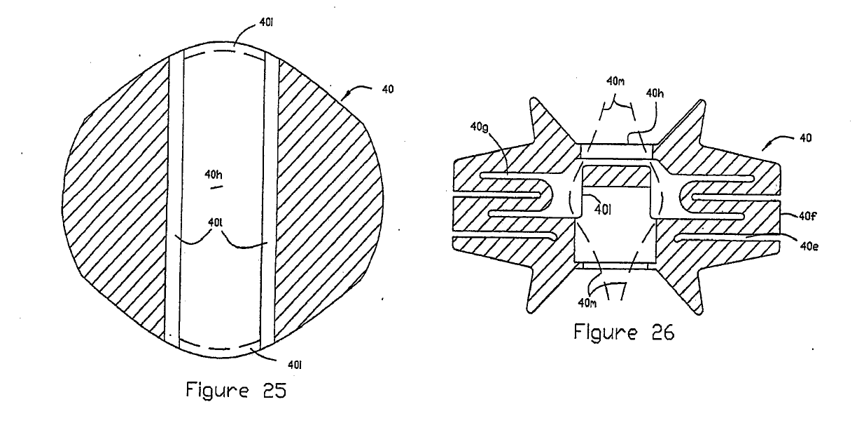

The bone diversion bar 40h creates channels 401 (FIG. 25) which promote relatively narrow bone growth along lines 40m to result in soft fusion. The cross-sectional area at the channel 401 is preferably within the range previously discussed.

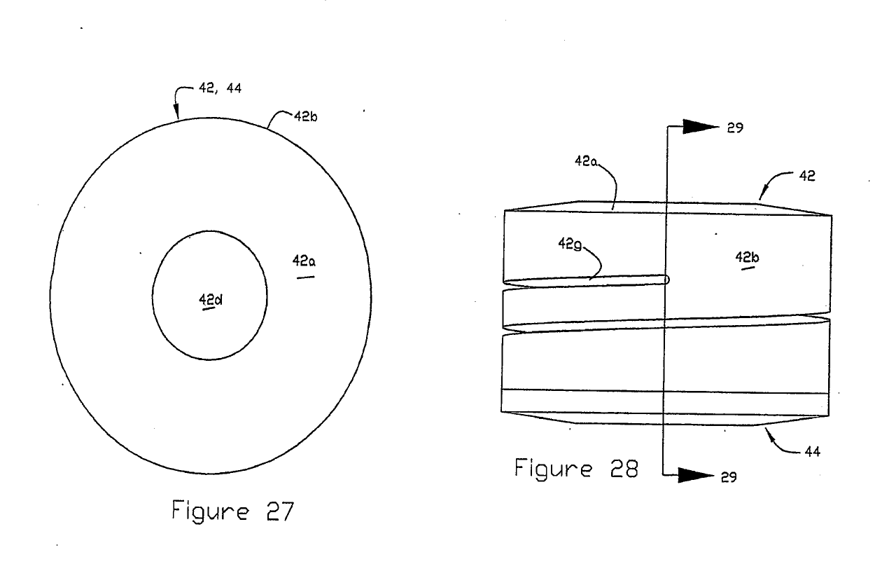

Another alternative hybrid intervertebral disc 42144 is illustrated in FIGS. 27-29 which includes an upper and lower section 42 and 44, respectively. The upper section 42 includes a top surface 42a, an exterior peripheral surface 42b, an inner surface 42c surrounding an open cylindrical core 42d. A ring-shaped inner cavity 42e, open at the lower end and forming an arch 42f at the upper end is formed in the upper section. The upper section is also formed with a helical ¾-1½ turn slot (or channel) 42g extending from the inner to the outer surfaces and through the cavity 42e as shown in FIG. 29. The slot is formed with stress relieving end openings 42h. The spiral slot 42g accommodates limited rotation about a vertical axis (e.g., about 3 degrees) and compression. The voids between the post and the cavity as well as the spiral slots accommodate the infiltration of soft tissue. The hollow core 42d will accommodate the infusion of bone and or soft tissue growth.

The inner surface 42i (FIG. 29) facing the cavity 42e is threaded at 42k for receiving the lower section 44. The lower section 44 is formed with an upwardly extending annular or donut-shaped post 44a extending into the cavity 42e. The lower section includes male threads 44b offset from an inner wall 44c thereof which threads cooperate with the threads 42k to join the lower section to the bottom of the upper section as shown. An outwardly extending flange 44d abuts an annular shoulder 42l to allow a surgeon to preset the compression of the spacer via the threads 42k/44b as will be apparent to those skilled in the art. The bottom surfaces 44d and 42n are arranged to engage the face of the lower vertebral body.

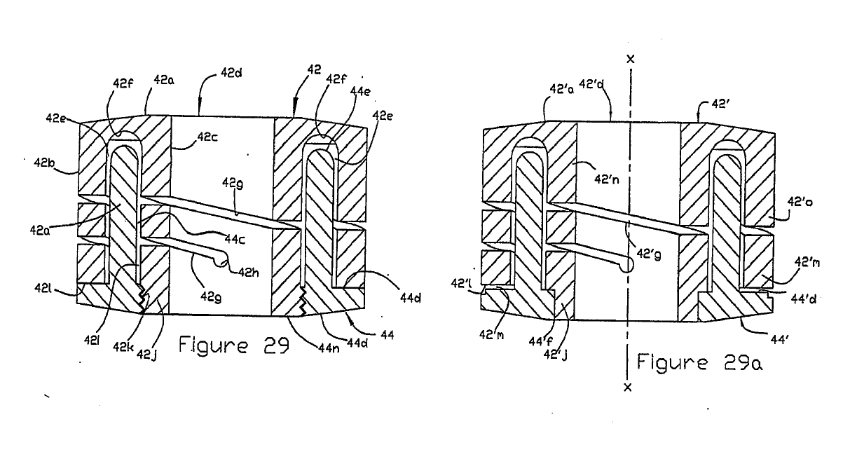

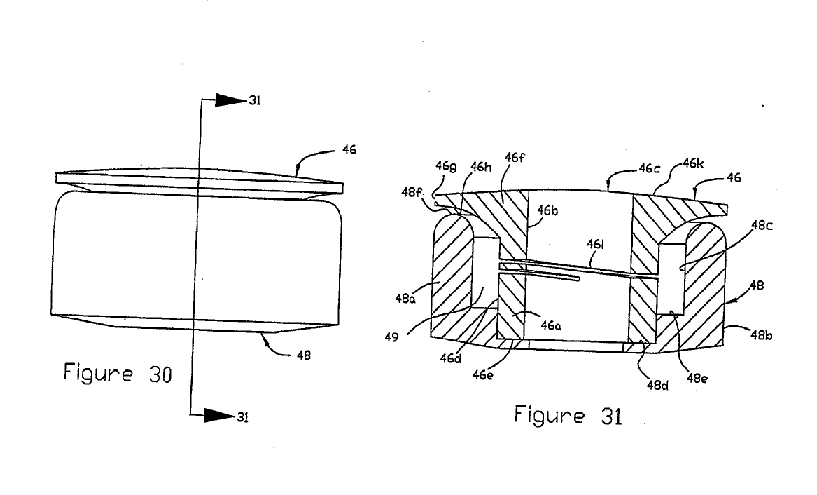

The abutting surfaces 44d and 42l will only transmit axial compressive and bending loads. This connection will only allow distractional, rotational and translational loads to be carried by the inner spring (formed by the inner cylindrical section 42j), softening the device in those motions. Excessive translations will contact surfaces 44c and 42i and then load the outer spring (formed by the outer cylindrical section 42m). The structure forming the inner and—outer springs is discussed in conjunction with FIG. 30.

The upper end 44e of the post 44a is arranged to abut the top 42f of the cavity to limit the compression and vertical articulation of the device.

FIG. 29a illustrates a slight variation of the disc of FIGS. 27-29. In this variation the threaded connection 42k/44b has been replaced with a weld at 44′f and the addition of a small gap 42′m, e.g., 0.010″ to 0.040″ between abutting surfaces 42′l and 44′d. As a result the inner spring, formed by the spiral slot 42′g in the inner wall 42′n, takes substantially all of the compressive load until the gap 42′m is closed. Then the outer spring, formed by the slot in the outer wall 42′o, assists with resisting the forces. This gap 42′m also serves another purpose. The outer spring accommodates only compressive loads (including bending), but not extraction or rotation about the longitudinal axis X-X. This arrangement softens the spacer for both loading conditions. The outer spring will also not absorb any translation until the gap 42′m is closed. This will allow motion more closely simulating that of a natural disc.

Another embodiment of a hybrid intervertebral disc 46148 is illustrated in the side elevational and cross-sectional views of FIGS. 30 and 31 with the understanding that the top plan view of the disc would be similar to that shown in FIG. 27. This spacer is formed with an upper section in the form of a cylindrical hub 46a, having an inner surface 46b surrounding an open central core 46c and an outer surface 46d. The hub extends upwardly from a flat bottom 46e to an outwardly flanged head portion 46f to a rim 46g. The lower section 48 is in the form of an annular post 48a having exterior and interior surfaces 48b and 48c, respectively, with the inner surface stair-stepped inwardly to form shelves 48d and 48e with the shelf 48e abutting the bottom 46e of the upper section. The convex, i.e., semicircular, upper end 48f of the post is arranged to abut the inner surface 46h of the flanged head 46f to stop the articulation of the hub when the device is subjected to excessive loads, while allowing limited rotation. The hub is formed with a ¾-1½ turn channel or slot 46i. The top and bottom surfaces 46k and 48g are arranged to engage the faces of the respective vertebral bodies to be supported.

The voids formed by the spinal slot and the space 49 between the outer and inner surfaces of the hub and the annular post, respectively, provide soft tissue ingrowth locations. The open core will allow bone and/or soft tissue ingrowth.

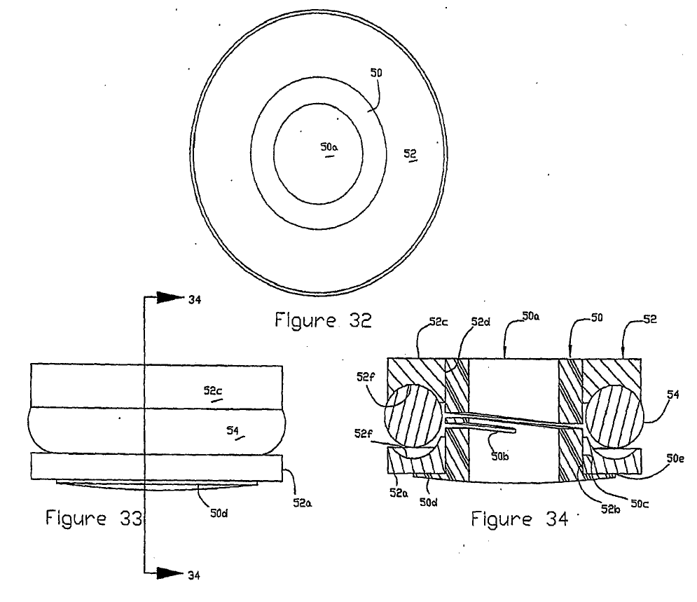

FIGS. 32-34 illustrate an additional embodiment of the present invention in the form of an inner cylindrical member 50 having an open core 50a adapted for bone and/or soft tissue ingrowth and a centrally located 1-1½ turn helical slot 50b adapted for soft tissue ingrowth. An annular outer member 52 includes a bottom portion 52a with its inner surface 52b secured, e.g., by welding, to the outer surface 50c of the inner member. The bottom 50d of the inner member forms an annular shelf 50e which sits under the bottom of the outer member portion 52a as shown. The top portion 52c of the outer member is secured at its inner surface 52d by welding, for example, to the outer surface along the top portion of the inner member. The top and bottom portions 52a and 52c are formed with concave mirror image surfaces 52e and 52f between which an articulation stopping ring 54 (circular in cross section), is positioned. The upper and lower surfaces of the 50/52 disc serve to engage the faces of the supported vertebral bodies.

The ring is preferably free floating within the space created by the surfaces 52e and 52d and smaller in diameter than the distance between such surfaces to allow the inner member to provide a limited amount of articulation, i.e., compression before making contact with both surfaces to stop the articulation resulting from an excessive load. The helical slot and the area surrounding the ring 54 are adapted for soft tissue ingrowth while the open core is adapted to accommodate bone and/or soft tissue ingrowth.

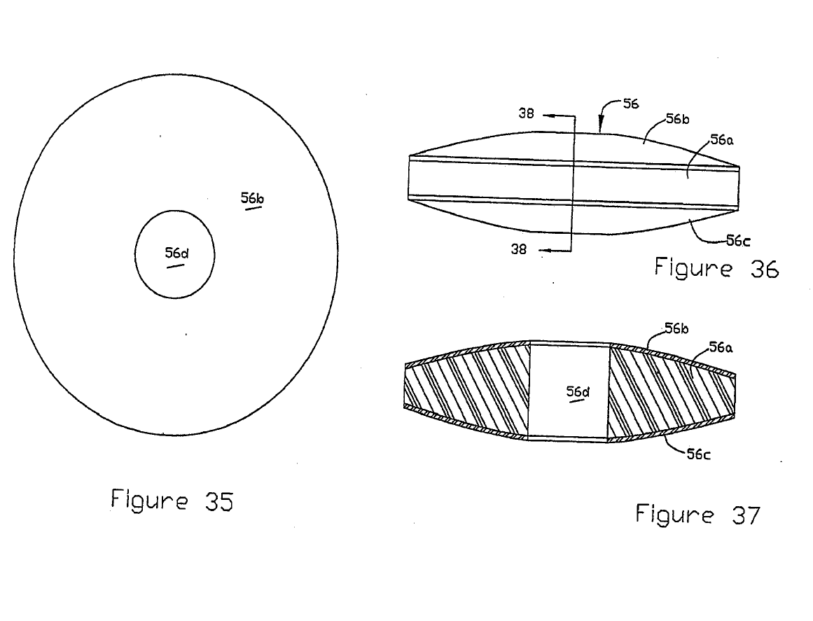

FIGS. 35-37 illustrate a modified disc 56 in which a suitable polymer 56a is enclosed by end plates or discs 56b and 56c with an open central core 56d for accommodating a bones strut to provide soft fusion. The spacer must have sufficient strength and stiffness (as discussed earlier) to support the adjacent vertebrae in their natural separated setting and yet under normal loads compress sufficiently to disrupt the bone struts within the open core to form one or more fibrous nonunion joints.

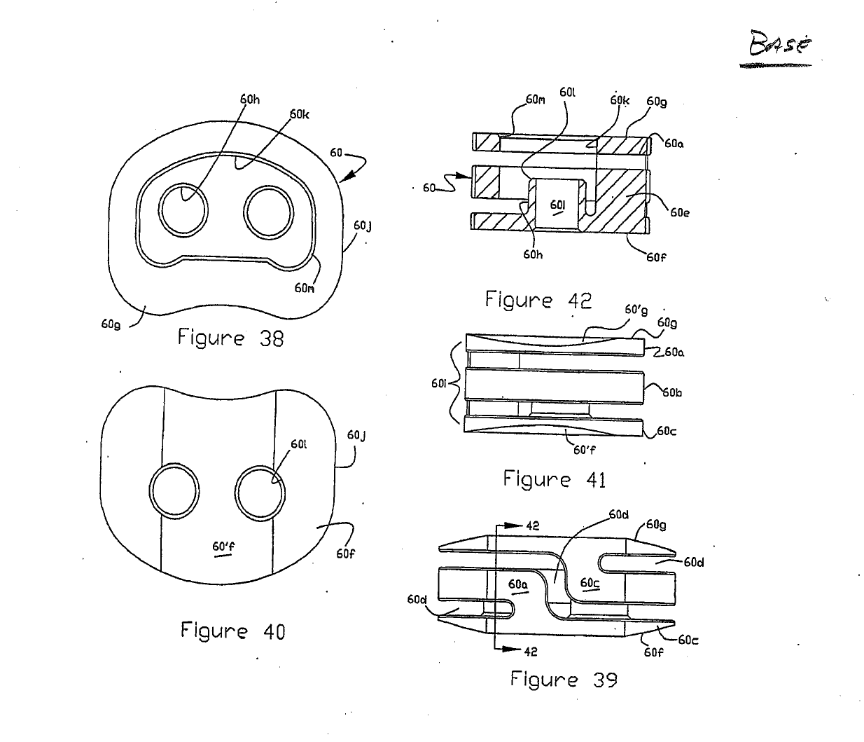

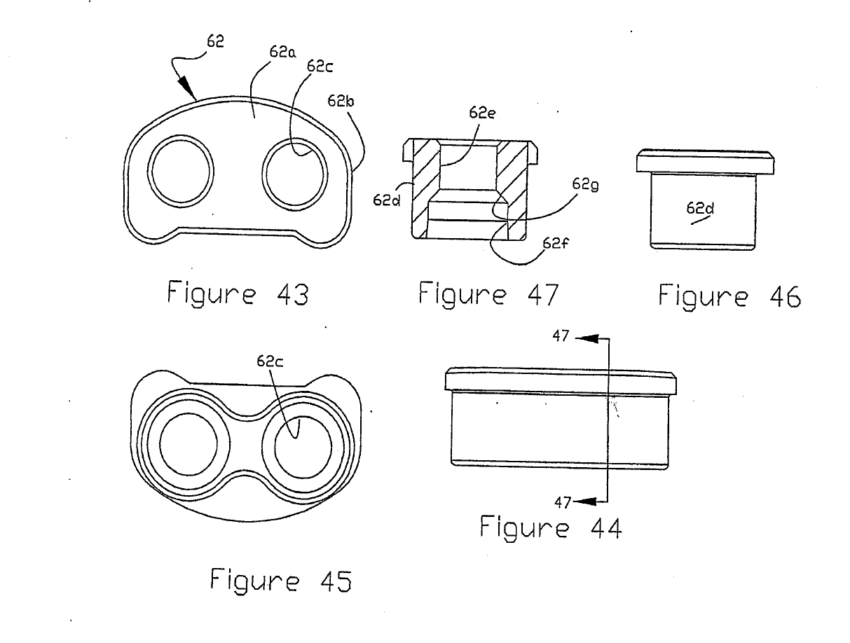

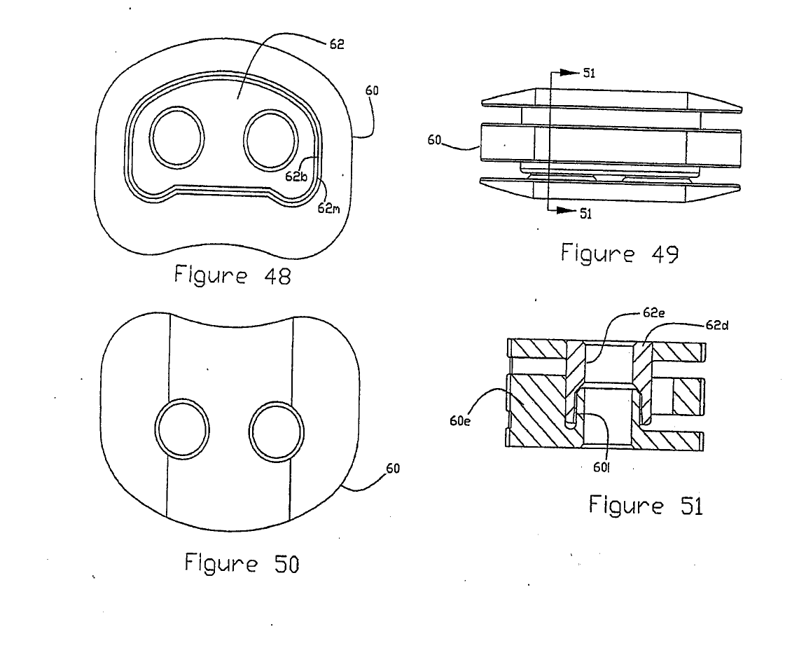

An additional two part disc, suitable for creating soft fusion, is illustrated in FIGS. 38-51 where FIGS. 38-42 show the bottom or base component 60, FIGS. 43-47 show the top or upper component 62, and FIGS. 48-51 show the assembled disc. The base component comprises upper and lower rings 60a and 60c separated via a partial ring 60d which is joined by bridged portions 60e to the upper and lower rings, as shown (FIGS. 42,43). A serpentine slot 60d extends through the base component accommodating the infusion of tissue and allowing limited axial and rotational motion between the upper and lower rings. Bottom and top walls 60f and 60g include centrally located concave portions 60′f and 60′g for engaging the exposed surfaces of the supported vertebral bodies. A pair of tubular posts 60h, defining open cores 60i, extend upwardly from the lower ring 60c. The outer surfaces of the rings define a peripheral wall 60j (FIG. 41) conforming generally to the kidney shape of the face of the vertebral bodies to be supported. A central section 60k surrounding the posts extends upwardly from the lower ring to an open top. A beveled surface 60m is formed at the upper end of the central section to provide a seat for a plate 62a of the top component to be described. In addition, an annular bevels 60l is formed on the outer surface at the top of tubular posts as shown to mate with a matching bevel on nipples carried by the upper component to limit axial motion as is illustrated in FIG. 51.

The upper component 62, shown in FIGS. 43-47, includes a cover plate 62a contoured to mate with the open end of section 60b via a matching beveled surface 62b. The plate is formed with circular openings 62c from which depend tubular nipples 62d defining stepped openings, the upper portion 62e thereof transitioning to a lower portion 62f via a bevel 62g. The inner surface of the lower portion 62f is arranged to encircle the outer wall of a respective post 60h with the bevel 62g being arranged to engage the post bevel 62l to limit the axial travel of the disc as is illustrated in FIG. 51. The posts 60h and the supplier 62d are sometimes referred to as an appendage The base and upper components as assembled are secured together along the beveled surfaces 60m and 62b, for example, by a TIG welding operation to fill in the area between the beveled surfaces. See FIG. 48. In the unlikely event of flexural element failure, the device will collapse and allow the beveled edges 62g and 62l to contact and center the device, limiting motion while stabilizing the disc.

The aligned openings 60i and 62e form bone accommodating channels to enable pseudoarthosis struts to form therein, which along with the mechanical characteristics of the disc, provide soft fusion as discussed.

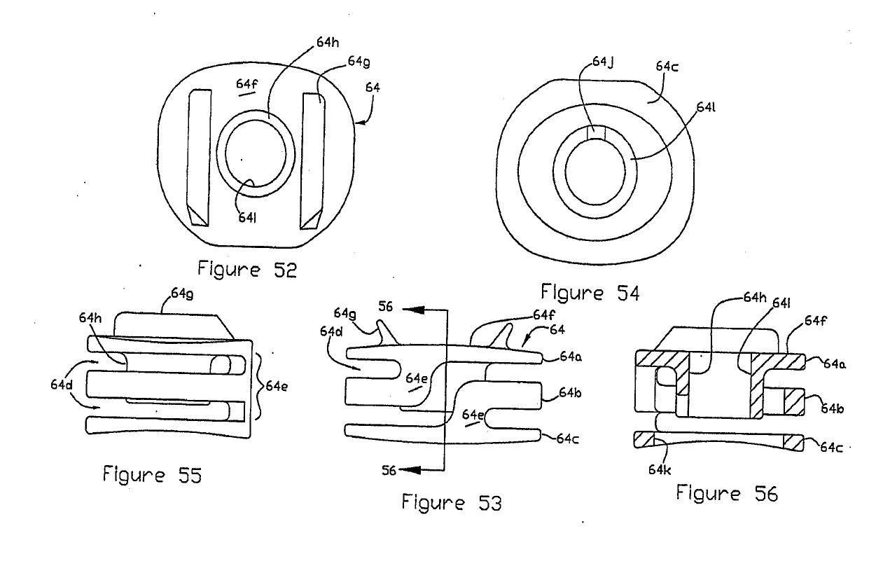

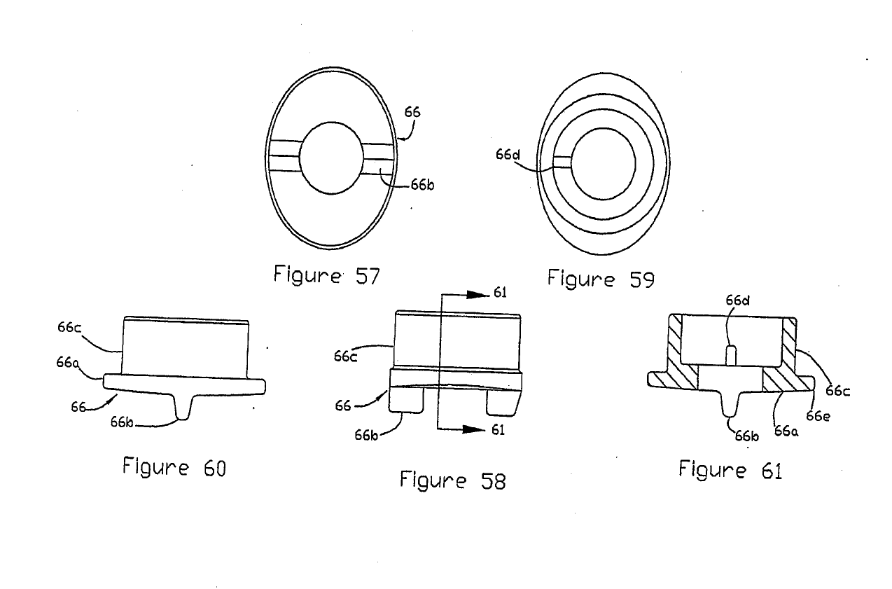

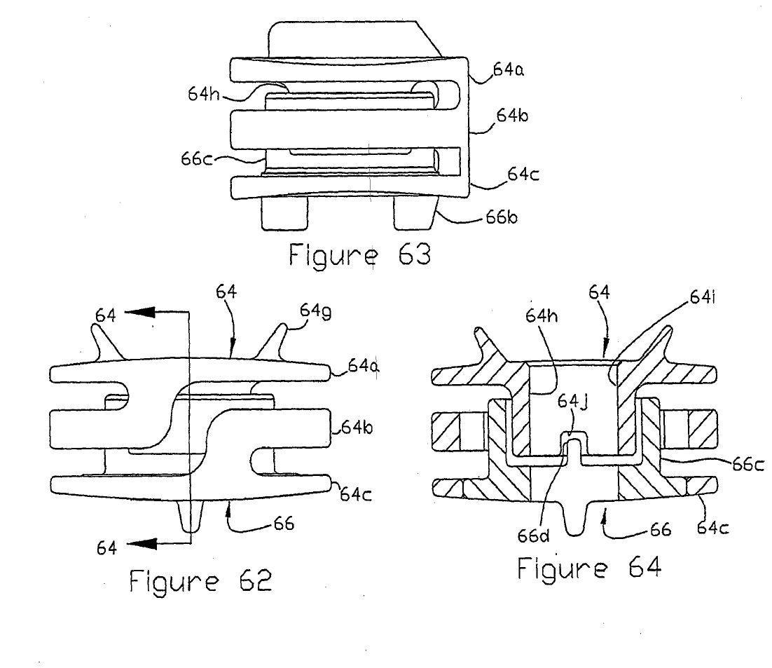

FIGS. 52-64 illustrate another two part disc in which FIGS. 52-56 show an upper (or inside) component; FIGS. 57-61 show a lower (or outside) component and FIGS. 62-63 show the assembled unit. The upper component 64 also comprises an upper and lower ring 61a and 64c with an intermediate partial ring 64b, separated from the upper and lower rings, by a serpentine tissue accommodating slot 64d and joined thereto by bridged segments 64e. As was discussed with respect to FIGS. 38-50 the slot accommodates the infusion of soft tissue and allows limited axial and rotational motion. A top wall 64f includes keels 64g and a hollow post 64h (defining a bone accommodation channel 64i) extends downwardly from the central section of the top ring and defines a notched keyway 64j in the bottom peripheral wall for cooperating with a mating upwardly projecting key formed on encompassing sleeve of the lower or outside component to be described for limiting the rotational mobility of the disc.

The lower component 66, shown in FIGS. 57-61, is formed with a base 66a supporting a pair of outwardly projecting spaced keels 66b, offset 90 degrees from the upper component keels, as is shown in FIG. 64 and an upwardly extending sleeve 66c arranged to surround the post 64h in the assembled condition. The base includes a radially inwardly projecting key 66d for mating with the keyway 64j. See FIG. 64. In the assembled condition, the two components are secured together, e.g., by welding the outer edge 66e of the base 66a to the inner edge 64k of the ring 64c as is indicated at 65 on FIG. 64.

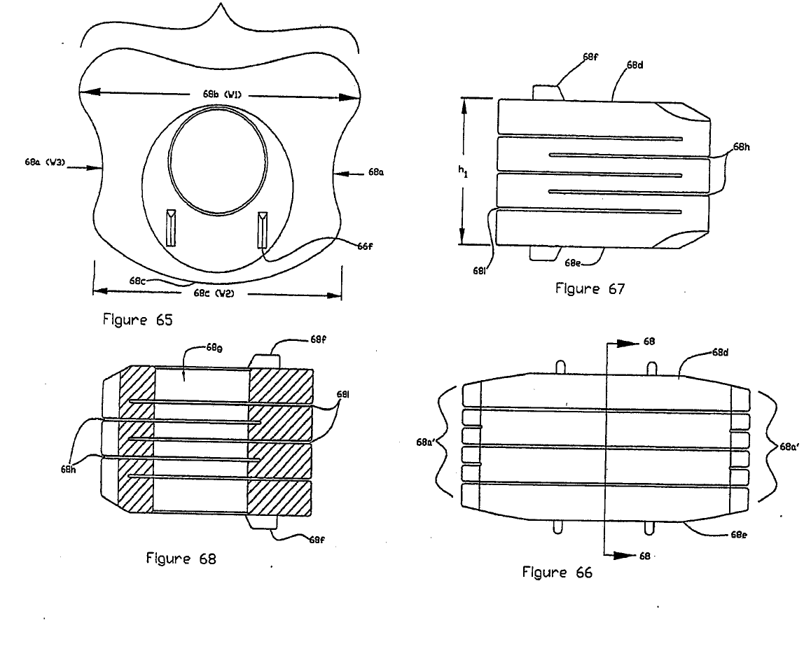

FIGS. 65-68 illustrate another embodiment of an anterior disc 68 in the general shape of a natural disc (e.g., kidney-shaped) as shown with pinched sides forming a relatively narrow midsection 68a, resulting in expanded or widened front and back wall areas 68b and 68c, respectively (in a horizontal cross-sectional view), and a narrower midsection (of the side walls 68′a) as is shown in FIG. 65. The disc further includes top and bottom surfaces 68d and 68e and keels 68f extending outwardly from the top and bottom surfaces. A centrally located core 68g accommodates bone growth to form living, not completely formed, bone struts. Interleaved lateral horizontal slots or slits 68h and 68i extend from the front and back walls, respectively, through the open core, as shown, to accommodate axial and binding loads and the infiltration of soft tissue. The slots 68h and 68i terminate a short distance from the back and front walls, respectively. As an example the widths W1, W2 at the front and rear expanded wall areas 68b and 68c and W3 at the narrow waist wall area, may be about 1.4, 1.2, and 0.95 inches, respectively, as is illustrated in FIG. 65. With the above dimensions in mind, the 68h and 68i slots may have a depth of about 0.012 to 0.014 inches and terminate about 0.250 and 0.135 inches from the back and front walls, respectively. The height h1 of the disc will vary depending upon its intended location. For example, h1 may vary between about 0.2 to 0.38 inches. Also, the number of slots may and probably will vary depending upon the height of the disc with the shorter discs having three slots while the higher discs will have five slots, for example.

As the disc flexes the widened front and back sections adjacent wall 68b and 68c overlying the slots, transition from level to level (vertically) compressing the slots these wall areas tend to widen out. This action allows these wider areas to transition the load to the next bend or level without fatiguing the disc material. By the same token, the narrower mid-section 68a allows more bending, but still without causing fatigue failure. The collapse of the slots with or without soft tissue infused therein serves to limit the compression of the disc due to excessive loads inhibiting fatigue failure.

FIGS. 69-72 illustrate an intervertebral disc 70 designed for posterior implantation. The disc (like the previously described discs) is formed of a suitable biocompatible material, such as Ti, stainless steel, etc. The disc includes a bulbous nose section 70a, with a threaded blind bore 70a for receiving an implantation tool (not shown) and a tail section 70b with side sections 70c extending between upper and lower vertebral body engaging surface: 70d and 70e. A central bone growth accommodating opening 70f is located between the side sections. The disc is elliptically shaped in an elevational and cross-sectional view as is shown in FIGS. 53 and 54. The disc is formed with fore and aft horizontal tissue accommodating slits or channels 70g which extend through the nose and tail sections and partially through the side sections as shown. A centrally located slit 70h extends through the side sections and into the nose section. The slits allow limited axial and bending motions. A centrally located aperature 70j accommodates the insertion of a wire for forming the slot 70h during the manufacturing operation The vertebrae engaging surfaces 70d and 70e are roughened, i.e., forming projecting pyramids, to provide bone attachment friendly surfaces.

FIG. 73 illustrates the placement of two of the posterior discs 70 on the face of an underlying vertebral body 8. As pointed out previously, the voids between and outside of the discs may be filled in with a material inhibiting bone growth.

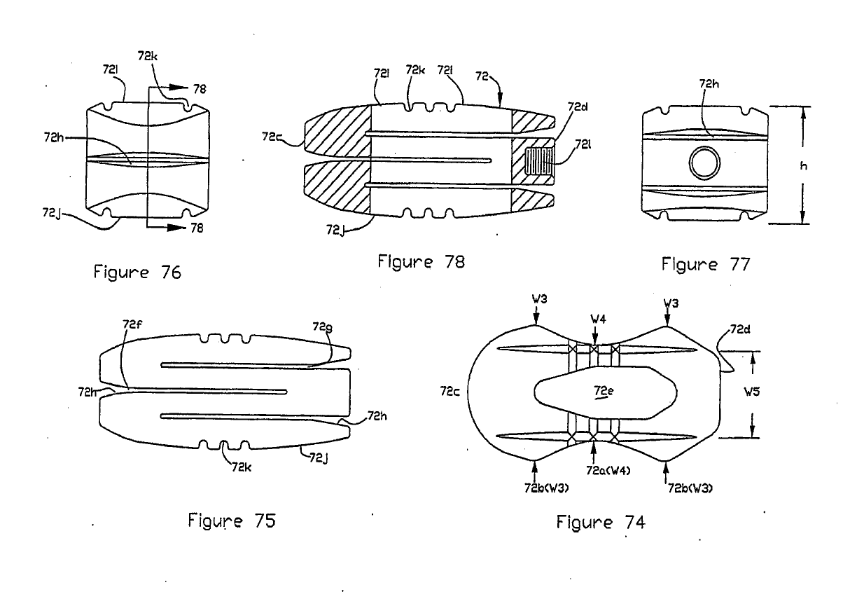

FIGS. 74-78 illustrate an additional intervertebral disc 72 designed for posterior insertion which, like the anterior disc of FIGS. 65-68, is formed with pinched side walls 72a at the center thereof and expanded or widened intermediate side wall sections 72b (adjacent the front and rear end walls 72c and 72d) for accommodating higher loads in the wider intermediate sections and increased bending along the center section. As an example, with an overall length of about 1.0″ and a height h of about 0.40″, the widths W3 of the widened areas may be about 0.48″ and the center narrower area W4 about 0.36″ and the width WS of the end 72d is about 0.25″. The disc defines a centralized elliptically shaped bone channel 72e with slots 72f and 72g extending from the front and back, respectively, through the side walls and core 72e, but terminating short of the rear and front walls, as shown. The outlet of the slots are tapered at 72h to accommodate bending stresses. The top and bottom surfaces 72i and 72j are formed with grooves 72k to enhance bone attachment. A threaded blind hole 72l is adapted to receive the threaded end of an insertion tool.

While providing various examples of intervertebral prosthetic discs and a method for accommodating the creation of soft fusion within the discs advances the state of this art, we now propose improvements to provide a superior prosthesis and method by filling the channels defined in the intervertebral prosthetic discs with a material which is less stiff than typical cortical bone including some cancellous bone used in the prior art devices. For example, a filler material having a flexural stiffness less than 10-12 GPa will improve the load compliance and flexibility of the intervertebral prosthesis.

Where a cortical/cancellous bone blend is to be used as the filler material, cancellous bone, which has a GPa of the order of 4 GPa, should comprise at least the predominate, if not, the sole constituent of the blend. This flexural stiffness is reported to be the average for cancellous bone. The use of such softer filler materials in the discs will allow for a more compliant and energy absorbing device even in the absence of a nonunion joint or pseudoarthosis. A softer filler material will in effect alleviate the need for a nonunion or pseudoarthosis by the formation of a more compliant yet stable fusion.

An interbody disc, in accordance with the present invention, has (a) upper and lower surfaces for engaging the faces of the adjacent vertebral bodies between which a failed natural disc has been partially or wholly removed, (b) an exterior wall and one or more generally vertically oriented continuous or discontinuous channels (c) a sufficient stiffness to support the separated vertebrae in substantially their naturally spaced relationship while allowing limited motion and flexibility when subjected to a predetermined load to alter the distance between the vertebrae and thereby transfer load and energy to the any material filling the channels or voids and (d) a bio-compatible filler material disposed within the channels, the filler material being compliant and softer than cortical bone, e.g., having a flexural stiffness of less than about 10-12 GPa. The filler material combined with the device characteristics—will dampen the loads and energy transfer prior to the device contacting on the internal stops which will then in turn prevent fatigue failure.

Where human bone is selected as the filler material, cancellous bone is the first choice. As a second choice cancellous and cortical bone can be blended with cancellous bone being the predominate portion of the blend such as a ratio of cancellous to cortical bone within the range of about 80% to 20% and preferably about 60%+ to 40%.

Other naturally harvested materials (either from the patient, a donor or an animal) suitable for use in the blend can include any substances softer than bone, such as portions of the removed disc. Morselized bone or bone weakened by gamma sterilization is more compliant then cortical bone and may also be useful as a filler material.

Bone graft substitutes, such as demineralized bone matrix (DBM), calcium sulfate dehydrate (CSD) ceramic-based bone graft extenders, are believed to be satisfactory filler materials. These will have a very low flexural modulus to allow device bending characteristics but will resist compressive forces when contained in a generally vertical channel. Recombinant Human Bone Morphogenetic Protein (rhBMP-2) liquid, Epidermal Growth Factor (EGF) liquid, Platelet Derived Growth Factor (PDGF), Fibroblast Growth Factors (FGFs), Parathyroid Hormone Related Peptide (PTHrp), Insulin-like Growth Factors (IGFs), and Transforming Growth Factor-Betas (i.e., TGF-B1), may also accompany suitable filler materials in order to induce a specific biological response such as bone or soft tissue activity. Another filler material candidate is polyetheretherketone (PEEK) with or without porosity. This synthetic material has mechanical properties very similar to those in cortical bone without porosity and very similar to cancellous bone when used with porosity. In addition, it is highly controllable and stable allowing the disc to be preassembled with the filler material at a factory site.

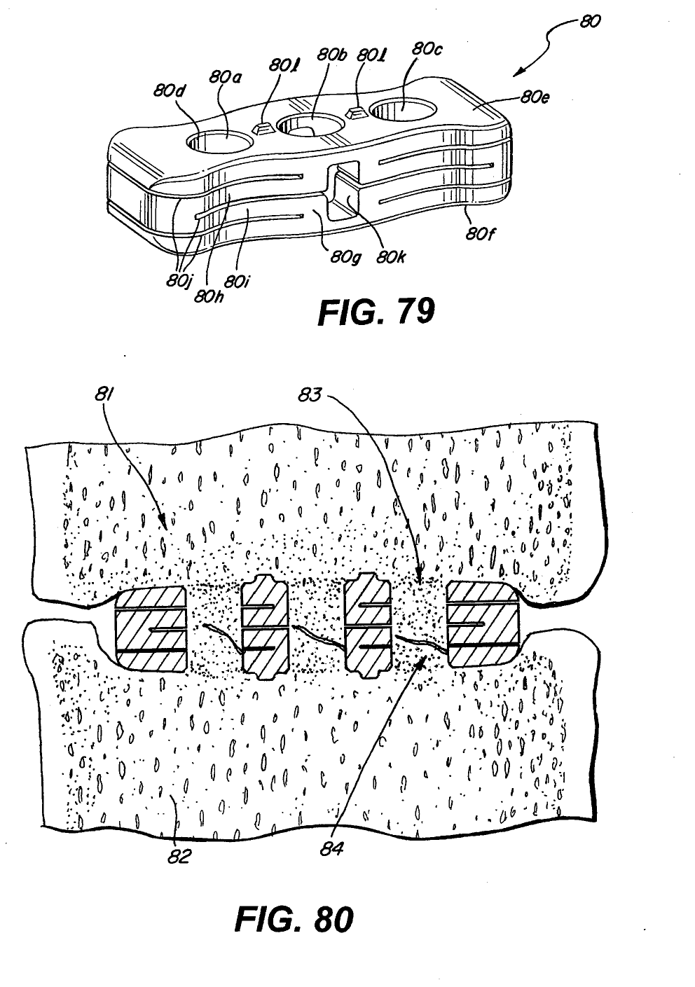

FIGS. 79 and 80 disclose, respectively, a prospective view of an elongated disc 80 formed from suitable biocompatible material with three circular vertical openings or channels 80a, 80b and 80c, having chamfered entrances 80d on the top and bottom of the disc. The body of the disc 80 has symmetrical top and bottom plates, 80e and 80f respectively, with sinusoidal sides 80g.

Between the top plate 80e and the bottom plate 80f are a pair of cantilevered spring plates 80h and 80i which are created by the respective overlapping slots 80i, to provide a controlled spring action.

A transverse rectangular opening 80k extends across the vertical opening 80b to enable bone growth not only from the top and bottom into the disc body through the vertical openings 80a, 80b and 80c, but also from either side through the transverse opening 80k thereby providing further securement of the disc 80.

Finally, a pair of keels 80l are respectively positioned on both the top and bottom surface of the disc 80 to provide a frictional contact with the corresponding surfaces of vertebral endplates.

FIG. 80 discloses a schematic cross sectional view of the disc 80 operatively positioned between vertebra 81 and 82 with a growth of bone into the respective vertical channels 80a, 80b and 80c. The filler material 83 has induced the bone fusion or growth in respectively each of the vertical channels and, while not shown, also in the transverse channel or opening 80k. The discontinuities 84 or “cracks” are indicative of an active patient or recipient where movement between the respective vertebra 81 and 82 has maintained openings that facilitate the spring movement of spring plates 80h and 80i.

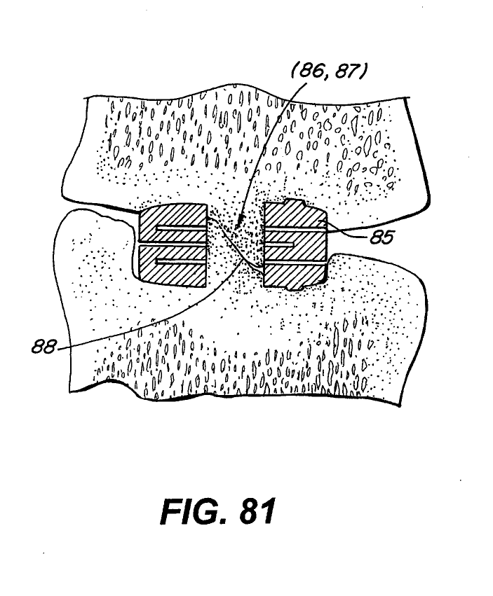

FIG. 81 represents a cross-sectional view of a disc 85 of a general type shown in FIGS. 65-68, however, with only two intermediate spring plates for insertion into this particular location along the spinal cord. Note, the specific vertebral height of the disc and number of spring plates in a disc will depend upon a desired size for a particular location along a spinal column. FIG. 81 is a representation of an actual cross-sectional view of the implanted disc 85 and a portion of a spinal column removed from a spine of a sheep as a test subject.

The filler material 86 was a mixture of cancellous and cortical bone and the bone growth 87 is shown with a discontinuity 88 indicative of movement of the spinal column by the sheep.

Referring now to FIGS. 20 and 21 as just an example of the present invention, the artificial intervertebral disc 10a includes upper and lower surfaces 11 and 12, respectively, for engaging the faces of adjacent vertebral bodies (such as 8 and 9 of FIG. 1) and an exterior wall 13a enclosing a vertically oriented core or channel 17. The structure and function of the several axial dampening plates 20 and 18 providing lateral channels 19 therebetween. The present invention is focused on the channel or core 17 which is filled with a filler material.

The filler material 83 may be inserted into the channel 17 at the surgical site by the surgeon or other attending personnel as shown in FIG. 21A. Some of the filler materials, e.g., PEEK, may be stable enough to be inserted at the factory. The number of channels for receiving the filler material is not limited to one and the channels need not be continuous or completely vertical. Potentially preassembled filler materials, such as PEEK, may be placed in, not only the generally vertical channels, but also the generally horizontal channels or voids, in order to dampen the device immediately prior to device contact or at the mechanical stops preventing fatigue failure. PEEK, with or without porosity, is available from Invibio, a wholly owned subsidiary of Victrex plc.

The specific resulting structure of our filler material within an artificial intervertebral disc after a period of time (six months or more) implanted within a patient will vary depending on the activity of the patient. For example, an older and/or less active patient may have a relatively sold infusion of bone growth, particularly in a central core opening of the intervertebral disc. A younger and/or more active patient will experience more motion applied to the intervertebral disc with appropriate flexion that will create and/or maintain openings or discontinuities in the bone growth and/or soft tissue infusion.

FIGS. 52-64 illustrate a two part disc in which FIGS. 52-56 show an upper (or inside) component and FIGS. 57-61 show a lower (or outside) component. FIGS. 62-63 show the assembled unit in a side elevational view and FIG. 64 is a cross-sectional view of the assembled unit. The upper component 64 comprises an upper and lower ring 61a and 64c with an intermediate partial ring 64b separated from the upper and lower rings, by a serpentine tissue accommodating slot 64d and joined thereto by bridged segments 64e. As was discussed with respect to FIGS. 38-50, the slot may accommodate the infusion of soft tissue and allow limited axial and rotational motion. A top wall 64f includes keels 64g and a hollow post 64h (defining a filler material accommodating top channel 64i) extends downwardly from the central section of the top ring and defines a notched keyway 64k in the bottom peripheral wall for cooperating with a mating upwardly projecting key formed on encompassing sleeve of the lower or outside component to be described for limiting the rotational mobility of the disc.

The lower component 66, shown in FIGS. 57-61, is formed with a base 66a supporting a pair of outwardly projecting spaced keels 66b, offset 90 degrees from the upper component keels, as is shown in FIG. 64A and an upwardly extending sleeve 66c (arranged to surround the post 64h in the assembled condition). The base defines a circular opening 66f which, together with the opening 64j, forms a channel for receiving the filler material 83. The base also includes a radially inwardly projecting key 66d for mating with the keyway 64k. See FIG. 64. In the assembled condition, the two components are secured together, e.g., by welding the outer edge 66e of the base 66a to the inner edge 64k of the ring 64c as is indicated at 65 on FIG. 64.

The prosthesis is completed by filling the channel 64j with an appropriate filler material 83 which is shown in FIG. 64A.

FIGS. 69-72 illustrate an intervertebral disc 70 designed for posterior implantation. The disc (like the previously described discs) is formed of a suitable biocompatible material, such as Ti, stainless steel, etc. The disc includes a bulbous nose section 70a, with a threaded blind bore 70a, for receiving an implantation tool (not shown) and a tail section 70b with side sections 70c extending between upper and lower vertebral body engaging surfaces 70d and 70e. A central filler material accommodating opening or channel 70f is located between the side sections. The disc is elliptically shaped in an elevational and cross-sectional view as is shown in FIGS. 53 and 54. The disc is formed with fore and aft horizontal tissue accommodating slits or channels 70g which extend through the nose and tail sections and partially through the side sections as shown. A centrally located slit 70h extends through the side sections and into the nose section. The slits allow limited axial and bending motions. A centrally located aperature 70j accommodates the insertion of a wire for forming the slot 70h during the manufacturing operation. The vertebrae engaging surfaces 70d and 70e are roughened, i.e., forming projecting pyramids, to provide bone attachment friendly surfaces. The channels 70f have been filled with a selected filter material 83 as is shown in the schematic perspective view of FIG. 73A. As shown in FIG. 73A a pair of discs can be positioned on the corresponding surfaces of vertebral endplates and the roughened surface 70 and 70e can assist in maintaining the respective disc placement while the filler material 83 facilitates the subsequent bone growth.

It is to be noted that the use of the term “adjacent” vertebral bodies includes the fifth lumbar vertebrae and the sacrum. It is also to be noted that the cross-sectional area of the channels to the total cross-sectional area of the disc may exceed the 35% preferred amount.

The method of the present invention entails the steps of a) providing a disc of the-type described herein b) filling the channel or channels with one of the filler materials described previously either at the surgical site or elsewhere, and c) inserting the completed disc between selected vertebral bodies.

There has been described a prosthetic intervertebral disc for restoring the motion between the supported vertebral bodies while enabling the formation of pseudo arthrosistic continuous or discontinuous bone struts having nonunion locations within the disc and between the supported bodies thereby providing a state of soft fusion and optionally accommodating the infusion of soft tissue within generally planar spaces within the disc. The disc may take many structural forms as is illustrated by the accompanying drawings. Variations and improvements to the soft fusion/hybrid disc of the present invention will undoubtedly occur to those skilled in the art without involving a departure from the invention as defined in the appended claims.

1. An intervertebral prosthesis comprising:
a) an interbody disc having upper and lower surfaces for engaging the faces of adjacent vertebral bodies between which a natural disc is to be partially or wholly removed, an exterior wall and one or more generally vertically oriented continuous or discontinous channels adapted to receive a filler material;
b) the disc having a stiffness sufficient to support the bodies in substantially their natural spaced relationship while allowing limited motion and being flexible enough when subjected to a predetermined load to alter the distance between the vertebrae engaging surfaces and transfer energy to any filler material within the channels;
c) the channel(s) being partially or completely filled with bio-compatible filler material having a flexural stiffness of less than about 12 GPa.

2. The prosthesis of claim 1 wherein the filler material has a flexural stiffness is less than about 10 GPa.

3. The prosthesis of claim 2 wherein the filler material has a flexural stiffness within the range of about 0-6 GPa.

4. The prosthesis of claim 1 wherein the filler material is predominately composed of cancellous bone.

5. The prosthesis of claim 2 wherein the filler material comprises about 60% to 70% of cancellus bone and about 30% to 40% cortical bone.

6. The prosthesis of claim 5 wherein the filler material comprises cancellous and cortical bone in the ratio of about 80% to 20%, respectively.

7. The prosthesis of claim 1, wherein the disc is further arranged to limit the movement thereof in the absence of fused bone within the channel(s) to an amount which is sustainable by the disc without resulting in fatigue failure during an anticipated life span.

8. The prosthesis of claim 1 wherein the disc further defines at least one generally horizontally oriented tissue channel extending from the filler material channel(s) through the exterior wall to accommodate the infusion of soft tissue therein.

9. The prosthesis of claim 1 wherein the filler material is PEEK with and/or without porosity.

10. The prosthesis of claim 1 wherein the filler material comprises 95% to 100% cancellous bone.

11. A method of replacing a damaged or failed natural spinal disc comprising:
a) providing an intervertebral disc having upper and lower surfaces for engaging the faces of the vertebral bodies between which the natural disc is to be partially or wholly removed, the disc having an exterior wall and one or more generally vertically oriented continuous or discontinuous channels adapted to receive a filler material and having sufficient stiffness, in the absence of the filler material to support the vertebral bodies in their spaced relationship while allowing limited motion and being flexible enough when subjected to a predetermined load to contract and transfer energy to the filler material within the channels;
b) filling the channels at least partially with a bio-compatible material having a flexural stiffness characteristic less than 10-12 GPa to form a completed prosthesis; and
c) inserting the prosthesis between the vertebral bodies between which a natural disc has been removed in whole or in part.

12. (canceled)

13. An intervertebral prosthesis comprising:
a) an interbody disc having upper and lower surfaces for engaging the faces of adjacent vertebral bodies between which a natural disc is to be partially or wholly removed, an exterior wall and one or more generally vertically oriented continuous or discontinuous channels adapted to receive a filler material;
b) the disc having a stiffness sufficient to support the bodies in substantially their natural spaced relationship while allowing limited motion and being flexible enough when subjected to a predetermined load to alter the distance between the vertebrae engaging surfaces and transfer energy to any filler material within the channels;
c) the channel(s) being partially or completely filled with bio-compatible filler such as cancellous bone or of an equivalent material of stiffness being less than cortical bone yet stiffer than soft tissue or a pseudoarthrosis.

14. An intervertebral prosthesis comprising:
an interbody disc having upper and lower surfaces for engaging the faces of adjacent vertebral bodies between which a natural disc is to be partially or wholly removed, an exterior wall and one or more generally vertically oriented continuous or discontinous channels adapted to receive a filler material, the interbody disc includes a plurality of generally sinuous oriented tissue channels formed by spaced openings extending across the interbody disc to provide a predetermined flexibility to receive compression and binding loads and to accommodate an infusion of soft tissue therein, the resilient compression and expansion of the tissue channels provide a connective transport of nutrients and waste material to and from the interbody disc when in situ,
wherein the channel(s) being partially or completely filled with bio-compatible filler material having a flexural stiffness of less than about 12 GPa, and the interbody disc having a stiffness sufficient to support the adjacent vertebral bodies in substantially their natural spaced relationship while allowing limited motion and being flexible enough when subjected to a predetermined load to alter the distance between the vertebrae engaging surfaces and transfer energy to filler material within the channels.

15. The intervertebral prosthesis of claim 14 wherein sinuous oriented tissue channels are formed in a continuous solid disc body member with spaced horizontal openings extending from a first side of the external wall to an opposite second side of the external wall.

16. The intervertebral prosthesis of claim 15 wherein the first side and second side of the external wall are offset by 90 degrees from a third side and an opposite fourth side of the external wall.

17. The intervertebral prostheses of claim 14 wherein a first set of sinuous oriented tissue channels are formed in a disc body member and a second set of sinuous oriented tissue channels are formed in the disc body member apart from the first set.

18. The intervertebral prostheses of claim 17 wherein one or more generally horizontal oriented continuous or discontinuous channels are adopted to receive a filler material to induce bone fusion or growth in each continuous or discontinuous channel.

19. The intervertebral prostheses of claim 17 wherein a generally horizontally oriented channel extends between and separates the respective first and second sets of sinuous oriented tissue channels.

20. The intervertebral prostheses of claim 19 wherein a plurality of vertically oriented continuous channels extend between the upper and lower surfaces of the interbody disc including a separate continuous channel through respectively, the first and second set of sinuous oriented tissue channels.

21. The intervertebral prostheses of claim 14 where three vertically oriented continuous channels extend between the upper and lower surfaces of the interbody disc to induce bone fusion or growth in each continuous channel.
